(19) United States
(12) Patent Application Publication
(10) Pub. No.: US 2010/0330097 A1
(43) Pub. Date: Dec. 30, 2010

(54) PREDICTING AMD WITH SNPS WITHIN OR NEAR C2, FACTOR B, PLEKHA1, HTRA1, PRELP, OR LOC387715

(76) Inventor: Gregory S. Hageman, Salt Lake City, UT (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 12/740,933

(22) PCT Filed: Nov. 3, 2008
(86) PCT No.: PCT/US08/82280

§ 371 Date: Aug. 13, 2010

Related U.S. Application Data
(60) Provisional application No. 60/984,702, filed on Nov. 1, 2007.

(51) Int. Cl. A61K 39/395; C12Q 1/68; A61K 38/14; A61K 31/7052; A61P 27/02

(52) U.S. Cl. 424/158.1; 435/6; 514/20.8; 514/44 A

(57) ABSTRACT

Gene polymorphisms and genetic profiles associated with an elevated or a reduced risk of a complement cascade dysregulation disease such as AMD are disclosed. Methods and reagents for determination of risk, diagnosis and treatment of such diseases are provided, as well as methods and reagents for determining sequence variants in the genome of an individual which facilitate assessment of risk for developing such diseases are provided.

### RELATED APPLICATIONS

This application claims the benefit of the priority date of U.S. Provisional Application No. 60/984,702, which was filed on Nov. 1, 2007, the contents of which are incorporated herein by reference in their entirety.

### FEDERALLY SPONSORED RESEARCH AND DEVELOPMENT

This invention was made with government support under NIH RO1 EY11515 and R24 EY017404, awarded by the National Institutes of Health. The government has certain rights in the invention.

### FIELD OF THE INVENTION

The invention relates to risk determination, diagnosis and prognosis of disorders such as age-related macular degeneration (AMD).

### BACKGROUND OF THE INVENTION

Age-related macular degeneration (AMD) is the leading cause of irreversible vision loss in the developed world, affecting approximately 15% of individuals over the age of 60. The prevalence of AMD increases with age: mild, or early, forms occur in nearly 30%, and advanced forms in about 7%, of the population that is 75 years and older. Clinically, AMD is characterized by a progressive loss of central vision attributable to degenerative changes that occur in the macula, a specialized region of the neural retina and underlying tissues. In the most severe, or exudative, form of the disease neovascular fronds derived from the choroidal vasculature breach Bruch's membrane and the retinal pigment epithelium (RPE) typically leading to detachment and subsequent degeneration of the retina.

Numerous studies have implicated inflammation in the pathobiology of AMD (Anderson et al. (2002) Am. J. Ophthalmol. 134:41 1-31; Hageman et al. (2001) Prog. Retin. Eye Res. 20:705-32; Mullins et al. (2000) Faseb J. 14:835-46; Johnson et al. (2001) Exp. Eye Res. 73:887-96; Crabb et al. (2002) PNAS 99:14682-7; Bok (2005) PNAS 102:7053-4). Dysfunction of the complement pathway may induce significant bystander damage to macular cells, leading to atrophy, degeneration, and the elaboration of choroidal neovascular membranes, similar to damage that occurs in other complement-mediated disease processes (Hageman et al. (2005) PNAS 102:7227-32: Morgan and Walport (1991) Immunol. Today 12:301-6; Kinoshita (1991) Immunol. Today 12:291-5; Holers and Thurman (2004) Mol. Immunol. 41: 147-52).

AMD, a late-onset complex disorder, appears to be caused and/or modulated by a combination of genetic and environmental factors. According to the prevailing hypothesis, the majority of AMD cases is not a collection of multiple single-gene disorders, but instead represents a quantitative phenotype, an expression of interaction of multiple susceptibility loci. The number of loci involved, the attributable risk conferred, and the interactions between various loci remain obscure, but significant progress has been made in determining the genetic contribution to these diseases. See, for example, U.S. Patent Application Publication No. 20070020647, U.S. Patent Application Publication No. 20060281120, International Publication No. WO 2008/013893, and U.S. Patent Application Publication No. 20080152659.

Thus, variations in several genes have been found to be correlated with AMD. These include the complement regulatory gene Complement Factor H (HF1/CFH) (see, for example, Hageman et al., 2005, Proc. Nat'l Acad Sci 102: 7227-32). Factor H is located on chromosome 1 among several other, closely linked regulators of the complement cascade in what is referred to as the Regulators of Complement Activation (RCA) locus. Deletions and other variations in other genes of the RCA locus (such as CFH-related 3 [FHR3] and CFH-related 1 [FHR1], among others) have also been correlated with AMD. See, for example, International Publication No. WO2008/008986, and Hughes et al., 2006, Nat Genet. 38:458-62. Sequence variations in other complement regulators, such as complement component C2 and Complement Factor B, which are closely linked on chromosome 6, have also been associated with AMD risk. See, for example, International Publication No. WO 2007/095185. Closely linked genes on chromosome 10, including LOC387715, HTRA1, and PLEKHA1 have also been shown to harbor sequence variations informative of AMD risk. See, for example, U.S. Patent Application Publication No. US 2006/0281120; International Publication No. WO 2007/044897; and International Publication No. WO 2008/013893.

Analysis of single polynucleotide polymorphisms (SNPs) is a powerful technique for diagnosis and/or determination of risk for disorders such as AMD.

### SUMMARY OF THE INVENTION

The invention arises, in part, from a high density, large sample size, genetic association study designed to detect genetic characteristics associated with complement cascade dysregulation diseases such as AMD. The study revealed a large number of new SNPs never before reported and a still larger number of SNPs (and/or combination of certain SNPs) which were not previously reported to be associated with risk for, or protection from, the disease. The invention disclosed herein thus relates to the discovery of polymorphisms that are associated with risk for development of age-related macular degeneration (AMD). The polymorphisms are found within or near genes such as complement component C2 (C2); Complement Factor B (Factor B); pleckstrin homology domain containing, family A (phosphoinositide binding specific) member 1 (PLEKHA1); HtrA serine peptidase 1 (HTRA1, also known as PRSS11); proline/arginine-rich and leucine-rich repeat protein (PRELP); and LOC387715. The informative value of many of the specific SNPs disclosed herein has never before been recognized or reported, as far as the inventor is aware. The invention provides methods of screening for individuals at risk of developing AMD and/or for predicting the likely progression of early- or mid-stage established disease and/or for predicting the likely outcome of a particular therapeutic or prophylactic strategy.

In one aspect, the invention provides a diagnostic method of determining an individual's propensity to complement dysregulation comprising screening (directly or indirectly) for the presence or absence of a genetic profile characterized by polymorphisms in the individual's genome associated with complement dysregulation, wherein the presence of said genetic profile is indicative of the individual's risk of complement dysregulation. The profile may reveal that the individual's risk is increased, or decreased, as the profile may evidence increased risk for, or increased protection from, developing AMD. A genetic profile associated with complement dysregulation comprises one or more, typically multiple, single nucleotide polymorphisms selected from Table 1 or Table 1A. In certain embodiments, a genetic profile associated with complement dysregulation comprises any combination of at least 2, at least 5, or at least 10 single nucleotide polymorphisms selected from Table 1 or Table 1A.

In one aspect, the invention provides a diagnostic method of determining an individual's propensity to develop, or for predicting the course of progression, of AMD, comprising screening (directly or indirectly) for the presence or absence of a genetic profile that includes one or more, typically multiple, single nucleotide polymorphisms selected from Table 1 and/or Table 1A, which are informative of an individual's (increased or decreased) risk for developing AMD. In one embodiment, the polymorphisms are selected from Table 1 or include at least one polymorphism selected from Table 1. In some embodiments, the genetic profile includes any combination of at least 2, at least 5, or at least 10 single nucleotide polymorphisms selected from Tables 1 and/or 1A.

In one embodiment, a method for determining an individual's propensity to develop or for predicting the course of progression of age-related macular degeneration, includes screening for a combination of at least one, typically multiple, predisposing polymorphism and at least one, typically multiple, protective polymorphism set forth in Tables 1 and 1A. For example, the method may comprise screening for at least rs4151671 (T: protective); rs2421018 (G: protective); rs3750847 (A: risk); and rs2253755 (G: risk). Risk polymorphisms indicate that an individual has increased susceptibility to development or progression of AMD relative to the control population. Protective polymorphisms indicate that the individual has a reduced likelihood of development or progression of AMD relative to the control population. Neutral polymorphisms do not segregate significantly with risk or protection, and have limited or no diagnostic or prognostic value. Additional, previously known informative polymorphisms may and typically will be included in the screen. For example, additional risk-associated polymorphisms may include rs1061170, rs203674, rs1061147, rs2274700, rs12097550, rs203674, a polymorphism in exon 22 of CFH (R1210C), rs9427661, rs9427662, rs10490924, rs11200638, rs2230199, rs2511989, rs3753395, rs1410996, rs393955, rs403846, rs1329421, rs10801554, rs12144939, rs12124794, rs2284664, rs16840422, and rs6695321. Additional protection-polymorphisms may include: rs800292, rs3766404, rs529825, rs641153, rs4151667, rs547154, and rs9332739. In one embodiment, the screening incorporates one or more polymorphisms from the RCA locus, such as those included in Table 3. In some embodiments, the screening incorporates one or more polymorphisms from other genes having genetic variations correlating with AMD risk, such as the genes and SNPs disclosed in Table 4.

In another embodiment, a method for determining an individual's propensity to develop or for predicting the course of progression of AMD includes screening additionally for deletions within the RCA locus (i.e., a region of DNA sequence located on chromosome one that extends from the Complement Factor H (CFH) gene through the CD46 gene (also known as the MCP gene, e.g., from CFH through complement factor 13B) that are associated with AMD risk or protection. An exemplary deletion that is protective of AMD is a deletion of at least portions of the FHR3 and FHR1 genes. See, e.g., Hageman et al., 2006, “Extended haplotypes in the complement factor H (CFH) and CFH-related (CFHR) family of genes protect against age-related macular degeneration: characterization, ethnic distribution and evolutionary implications,” Ann Med. 38:592-604 and U.S. Patent Application Publication No. US 2008/152659.

The methods may include inspecting a data set indicative of genetic characteristics previously derived from analysis of the individual's genome. A data set of genetic characteristics of the individual may include, for example, a listing of single nucleotide polymorphisms in the individual's genome or a complete or partial sequence of the individual's genomic DNA. Alternatively, the methods include obtaining and analyzing a nucleic acid sample (e.g., DNA or RNA) from an individual to determine whether the DNA contains informative polymorphisms, such as by combining a nucleic acid sample from the subject with one or more polynucleotide probes capable of hybridizing selectively to a nucleic acid carrying the polymorphism. In another embodiment, the methods include obtaining a biological sample from the individual and analyzing the sample from the individual to determine whether the individual's proteome contains an allelic variant isoform that is a consequence of the presence of a polymorphism in the individual's genome.

In another aspect, the invention provides a method of treating, preventing, or delaying development of symptoms of AMD in an individual (e.g., an individual in whom a genetic profile indicative of elevated risk of developing AMD is detected), comprising prophylactically or therapeutically treating an individual identified as having a genetic profile including one or more single nucleotide polymorphisms selected from Table 1 and/or Table 1A.

In one embodiment, the method of treating or preventing AMD in an individual includes prophylatically or therapeutically treating the individual by administering a composition including a Factor H polypeptide. The Factor H polypeptide may be a wild type Factor H polypeptide or a variant Factor H polypeptide. The Factor H polypeptide may be a Factor H polypeptide with a sequence encoded by a protective or neutral allele. In one embodiment, the Factor H polypeptide is encoded by a Factor H protective haplotype. A protective Factor H haplotype can encode an isoleucine residue at amino acid position 62 and/or an amino acid other than a histidine at amino acid position 402. For example, a Factor H polypeptide can comprise an isoleucine residue at amino acid position 62, a tyrosine residue at amino acid position 402, and/or an arginine residue at amino acid position 1210. Exemplary Factor H protective haplotypes include the H2 haplotype or the H4 haplotype. Alternatively, the Factor H polypeptide may be encoded by a Factor H neutral haplotype. A neutral haplotype encodes an amino acid other than an isoleucine at amino acid position 62 and an amino acid other than a histidine at amino acid position 402. Exemplary Factor H neutral haplotypes include the H3 haplotype or the H5 haplotype. For details on therapeutic forms of CFH, and how to make and use them, see U.S. Patent Application Publication No. US 2007/0060247, the disclosure of which is incorporated herein by reference.

In some embodiments, the method of treating or preventing AMD in an individual includes prophylactically or therapeutically treating the individual by inhibiting HTRA1 in the individual. HTRA1 can be inhibited, for example, by administering an antibody or other protein (e.g. an antibody variable domain, an addressable fibronectin protein, etc.) that binds HTRA1. Alternatively, HTRA1 can be inhibited by administering a nucleic acid inhibiting HTRA1 expression or activity, such as an inhibitory RNA, a nucleic acid encoding an inhibitory RNA, an antisense nucleic acid, or an aptamer, or by administering a small molecule that interferes with HTRA1 activity (e.g. an inhibitor of the protease activity of HTRA1).

In other embodiments, the method of treating or preventing AMD in an individual includes prophylactically or therapeutically treating the individual by inhibiting Factor B and/or C2 in the individual. Factor B can be inhibited, for example, by administering an antibody or other protein (e.g., an antibody variable domain, an addressable fibronectin protein, etc.) that binds Factor B. Alternatively, Factor B can be inhibited by administering a nucleic acid inhibiting Factor B expression or activity, such as an inhibitory RNA, a nucleic acid encoding an inhibitory RNA, an antisense nucleic acid, or an aptamer, or by administering a small molecule that interferes with Factor B activity (e.g., an inhibitor of the protease activity of Factor B). C2 can be inhibited, for example, by administering an antibody or other protein (e.g., an antibody variable domain, an addressable fibronectin protein, etc.) that binds C2. Alternatively, C2 can be inhibited by administering a nucleic acid inhibiting C2 expression or activity, such as an inhibitory RNA, a nucleic acid encoding an inhibitory RNA, an antisense nucleic acid, or an aptamer, or by administering a small molecule that interferes with C2 activity (e.g., an inhibitor of the protease activity of C2).

In another aspect, the invention provides detectably labeled oligonucleotide probes or primers for hybridization with DNA sequence in the vicinity of at least one polymorphism to facilitate identification of the base present in the individual's genome. In one embodiment, a set of oligonucleotide primers hybridizes adjacent to at least one polymorphism disclosed herein for inducing amplification thereof, thereby facilitating sequencing of the region and determination of the base present in the individual's genome at the sites of the polymorphism. Preferred polymorphisms for detection include the polymorphisms listed in Table 1 or 1A. Further, one of skill in the art will appreciate that other methods for detecting polymorphisms are well known in the art.

In another aspect, the invention relates to a healthcare method that includes authorizing the administration of, or authorizing payment for the administration of a diagnostic assay to determine an individual's susceptibility for development or progression of AMD. The method includes screening for the presence or absence of a genetic profile that includes one or more SNPs selected from Table 1 or 1A.

### DETAILED DESCRIPTION OF THE INVENTION

### I. Definitions and Conventions

The term “polymorphism” refers to the occurrence of two or more genetically determined alternative sequences or alleles in a population. Each divergent sequence is termed an allele, and can be part of a gene or located within an intergenic or non-genic sequence. A diallelic polymorphism has two alleles, and a triallelic polymorphism has three alleles. Diploid organisms can contain two alleles and may be homozygous or heterozygous for allelic forms. The first identified allelic form is arbitrarily designated the reference form or allele; other allelic forms are designated as alternative or variant alleles. The most frequently occurring allelic form in a selected population is typically referred to as the wild-type form.

A “polymorphic site” is the position or locus at which sequence divergence occurs at the nucleic acid level and is sometimes reflected at the amino acid level. The polymorphic region or polymorphic site refers to a region of the nucleic acid where the nucleotide difference that distinguishes the variants occurs, or, for amino acid sequences, a region of the amino acid sequence where the amino acid difference that distinguishes the protein variants occurs. A polymorphic site can be as small as one base pair, often termed a “single nucleotide polymorphism” (SNP). The SNPs can be any SNPs in loci identified herein, including intragenic SNPs in exons, introns, or upstream or downstream regions of a gene, as well as SNPs that are located outside of gene sequences. Examples of such SNPs include, but are not limited to, those provided in the Tables herein below.

Individual amino acids in a sequence are represented herein as AN or NA, wherein A is the amino acid in the sequence and N is the position in the sequence. In the case that position N is polymorphic, it is convenient to designate the more frequent variant as A₁N and the less frequent variant as NA₂. Alternatively, the polymorphic site, N, is represented as A₁NA₂, wherein A₁ is the amino acid in the more common variant and A₂ is the amino acid in the less common variant. Either the one-letter or three-letter codes are used for designating amino acids (see Lehninger, Biochemistry 2nd ed., 1975, Worth Publishers, Inc. New York, N.Y.: pages 73-75, incorporated herein by reference). For example, 150V represents a single-amino-acid polymorphism at amino acid position 50 of a given protein, wherein isoleucine is present in the more frequent protein variant in the population and valine is present in the less frequent variant.

Similar nomenclature may be used in reference to nucleic acid sequences. In the Tables provided herein, each SNP is depicted by “N₁/N₂” where N₁ is a nucleotide present in a first allele referred to as Allele 1, and N₂ is another nucleotide present in a second allele referred to as Allele 2. It will be clear to those of skill in the art that in a double-stranded form, the complementary strand of each allele will contain the complementary base at the polymorphic position.

The term “genotype” as used herein denotes one or more polymorphisms of interest found in an individual, for example, within a gene of interest. Diploid individuals have a genotype that comprises two different sequences (heterozygous) or one sequence (homozygous) at a polymorphic site.

The term “haplotype” refers to a DNA sequence comprising one or more polymorphisms of interest contained on a subregion of a single chromosome of an individual. A haplotype can refer to a set of polymorphisms in a single gene, an intergenic sequence, or in larger sequences including both gene and intergenic sequences, e.g., a collection of genes, or of genes and intergenic sequences. For example, a haplotype can refer to a set of polymorphisms on chromosome 10 near the PLEKHA1, LOC387715 and HTRA1 genes, e.g. within the genes and/or within intergenic sequences (i.e., intervening intergenic sequences, upstream sequences, and downstream sequences that are in linkage disequilibrium with polymorphisms in the genic region). The term “haplotype” can refer to a set of single nucleotide polymorphisms (SNPs) found to be statistically associated on a single chromosome. A haplotype can also refer to a combination of polymorphisms (e.g., SNPs) and other genetic markers (e.g., a deletion) found to be statistically associated on a single chromosome. A haplotype, for instance, can also be a set of maternally inherited alleles, or a set of paternally inherited alleles, at any locus.

The term “genetic profile,” as used herein, refers to a collection of one or more single nucleotide polymorphisms including a polymorphism shown in Table 1 (AMD), optionally in combination with other genetic characteristics such as deletions, additions or duplications, and optionally combined with other SNPs associated with AMD risk or protection, including but not limited to those in Tables 3 and 4. Thus, a genetic profile, as the phrase is used herein, is not limited to a set of characteristics defining a haplotype, and may include SNPs from diverse regions of the genome. For example, a genetic profile for AMD includes one or a subset of single nucleotide polymorphisms selected from Table 1, optionally in combination with other genetic characteristics associated with AMD. It is understood that while one SNP in a genetic profile may be informative of an individual's increased or decreased risk (i.e., an individual's propensity or susceptibility) to develop a complement-related disease such as AMD, more than one SNP in a genetic profile may and typically will be analyzed and will be more informative of an individual's increased or decreased risk of developing a complement-related disease. A genetic profile may include at least one SNP disclosed herein in combination with other polymorphisms or genetic markers (e.g., a deletion) and/or environmental factors (e.g., smoking or obesity) known to be associated with AMD. In some cases, a SNP may reflect a change in regulatory or protein coding sequences that change gene product levels or activity in a manner that results in increased likelihood of development of disease. In addition, it will be understood by a person of skill in the art that one or more SNPs that are part of a genetic profile maybe in linkage disequilibrium with, and serve as a proxy or surrogate marker for, another genetic marker or polymorphism that is causative, protective, or otherwise informative of disease.

The term “gene,” as used herein, refers to a region of a DNA sequence that encodes a polypeptide or protein, intronic sequences, promoter regions, and upstream (i.e., proximal) and downstream (i.e., distal) non-coding transcription control regions (e.g., enhancer and/or repressor regions).

The term “allele,” as used herein, refers to a sequence variant of a genetic sequence (e.g., typically a gene sequence as described hereinabove, optionally a protein coding sequence). For purposes of this application, alleles can but need not be located within a gene sequence. Alleles can be identified with respect to one or more polymorphic positions such as SNPs, while the rest of the gene sequence can remain unspecified. For example, an allele may be defined by the nucleotide present at a single SNP, or by the nucleotides present at a plurality of SNPs. In certain embodiments of the invention, an allele is defined by the genotypes of at least 1, 2, 4, 8 or 16 or more SNPs, (including those provided in Tables 1 and 1A below) in a gene.

A “causative” SNP is a SNP having an allele that is directly responsible for a difference in risk of development or progression of AMD. Generally, a causative SNP has an allele producing an alteration in gene expression or in the expression, structure, and/or function of a gene product, and therefore is most predictive of a possible clinical phenotype. One such class includes SNPs falling within regions of genes encoding a polypeptide product, i.e. “coding SNPs” (cSNPs). These SNPs may result in an alteration of the amino acid sequence of the polypeptide product (i.e., non-synonymous codon changes) and give rise to the expression of a defective or other variant protein. Furthermore, in the case of nonsense mutations, a SNP may lead to premature termination of a polypeptide product. Such variant products can result in a pathological condition, e.g., genetic disease. Examples of genes in which a SNP within a coding sequence causes a genetic disease include sickle cell anemia and cystic fibrosis.

Causative SNPs do not necessarily have to occur in coding regions; causative SNPs can occur in, for example, any genetic region that can ultimately affect the expression, structure, and/or activity of the protein encoded by a nucleic acid. Such genetic regions include, for example, those involved in transcription, such as SNPs in transcription factor binding domains, SNPs in promoter regions, in areas involved in transcript processing, such as SNPs at intron-exon boundaries that may cause defective splicing, or SNPs in mRNA processing signal sequences such as polyadenylation signal regions. Some SNPs that are not causative SNPs nevertheless are in close association with, and therefore segregate with, a disease-causing sequence. In this situation, the presence of a SNP correlates with the presence of or predisposition to, or an increased risk in developing the disease. These SNPs, although not causative, are nonetheless also useful for diagnostics, disease predisposition screening, and other uses.

An “informative” or “risk-informative” SNP refers to any SNP whose sequence in an individual provides information about that individual's relative risk of development or progression of AMD. An informative SNP need not be causative. Indeed, many informative SNPs have no apparent effect on any gene product, but are in linkage disequilibrium with a causative SNP. In such cases, as a general matter, the SNP is increasingly informative when it is more tightly in linkage disequilibrium with a causative SNP. For various informative SNPs, the relative risk of development or progression of AMD is indicated by the presence or absence of a particular allele and/or by the presence or absence of a particular diploid genotype.

The term “linkage” refers to the tendency of genes, alleles, loci, or genetic markers to be inherited together as a result of their location on the same chromosome or as a result of other factors. Linkage can be measured by percent recombination between the two genes, alleles, loci, or genetic markers. Some linked markers may be present within the same gene or gene cluster.

In population genetics, linkage disequilibrium is the non-random association of alleles at two or more loci, not necessarily on the same chromosome. It is not the same as linkage, which describes the association of two or more loci on a chromosome with limited recombination between them. Linkage disequilibrium describes a situation in which some combinations of alleles or genetic markers occur more or less frequently in a population than would be expected from a random formation of haplotypes from alleles based on their frequencies. Non-random associations between polymorphisms at different loci are measured by the degree of linkage disequilibrium (LD). The level of linkage disequilibrium is influenced by a number of factors including genetic linkage, the rate of recombination, the rate of mutation, random drift, non-random mating, and population structure. “Linkage disequilibrium” or “allelic association” thus means the preferential association of a particular allele or genetic marker with another specific allele or genetic marker more frequently than expected by chance for any particular allele frequency in the population. A marker in linkage disequilibrium with an informative marker, such as one of the SNPs listed in Tables I or IA can be useful in detecting susceptibility to disease. A SNP that is in linkage disequilibrium with a causative, protective, or otherwise informative SNP or genetic marker is referred to as a “proxy” or “surrogate” SNP. A proxy SNP may be in at least 50%, 60%, or 70% in linkage disequilibrium with the causative SNP, and preferably is at least about 80%, 90%, and most preferably 95%, or about 100% in LD with the genetic marker.

A “nucleic acid,” “polynucleotide,” or “oligonucleotide” is a polymeric form of nucleotides of any length, may be DNA or RNA, and may be single- or double-stranded. The polymer may include, without limitation, natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, 5-methylcytidine, C5-bromouridine, C5-fluorouridine, C5-iodouridine, C5-propynyl-uridine, C5-propynyl-cytidine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages). Nucleic acids and oligonucleotides may also include other polymers of bases having a modified backbone, such as a locked nucleic acid (LNA), a peptide nucleic acid (PNA), a threose nucleic acid (TNA) and any other polymers capable of serving as a template for an amplification reaction using an amplification technique, for example, a polymerase chain reaction, a ligase chain reaction, or non-enzymatic template-directed replication.

Oligonucleotides are usually prepared by synthetic means. Nucleic acids include segments of DNA, or their complements spanning any one of the polymorphic sites shown in the Tables provided herein. Except where otherwise clear from context, reference to one strand of a nucleic acid also refers to its complement strand. The segments are usually between 5 and 100 contiguous bases, and often range from a lower limit of 5, 10, 12, 15, 20, or 25 nucleotides to an upper limit of 10, 15, 20, 25, 30, 50 or 100 nucleotides (where the upper limit is greater than the lower limit). Nucleic acids between 5-10, 5-20, 10-20, 12-30, 15-30, 10-50, 20-50 or 20-100 bases are common. The polymorphic site can occur within any position of the segment. The segments can be from any of the allelic forms of DNA shown in the Tables provided herein.

“Hybridization probes” are nucleic acids capable of binding in a base-specific manner to a complementary strand of nucleic acid. Such probes include nucleic acids and peptide nucleic acids. Hybridization is usually performed under stringent conditions which are known in the art. A hybridization probe may include a “primer.”

The term “primer” refers to a single-stranded oligonucleotide capable of acting as a point of initiation of template-directed DNA synthesis under appropriate conditions, in an appropriate buffer and at a suitable temperature. The appropriate length of a primer depends on the intended use of the primer, but typically ranges from 15 to 30 nucleotides. A primer sequence need not be exactly complementary to a template, but must be sufficiently complementary to hybridize with a template. The term “primer site” refers to the area of the target DNA to which a primer hybridizes. The term “primer pair” means a set of primers including a 5′ upstream primer, which hybridizes to the 5′ end of the DNA sequence to be amplified and a 3′ downstream primer, which hybridizes to the complement of the 3′ end of the sequence to be amplified.

The nucleic acids, including any primers, probes and/or oligonucleotides can be synthesized using a variety of techniques currently available, such as by chemical or biochemical synthesis, and by in vitro or in vivo expression from recombinant nucleic acid molecules, e.g., bacterial or retroviral vectors. For example, DNA can be synthesized using conventional nucleotide phosphoramidite chemistry and the instruments available from Applied Biosystems, Inc. (Foster City, Calif.); DuPont (Wilmington, Del.); or Milligen (Bedford, Mass.). When desired, the nucleic acids can be labeled using methodologies well known in the art such as described in U.S. Pat. Nos. 5,464,746; 5,424,414; and 4,948,882 all of which are herein incorporated by reference. In addition, the nucleic acids can comprise uncommon and/or modified nucleotide residues or non-nucleotide residues, such as those known in the art.

An “isolated” nucleic acid molecule, as used herein, is one that is separated from nucleotide sequences which flank the nucleic acid molecule in nature and/or has been completely or partially purified from other biological material (e.g., protein) normally associated with the nucleic acid. For instance, recombinant DNA molecules in heterologous organisms, as well as partially or substantially purified DNA molecules in solution, are “isolated” for present purposes.

The term “target region” refers to a region of a nucleic acid which is to be analyzed and usually includes at least one polymorphic site.

“Stringent” as used herein refers to hybridization and wash conditions at 50° C. or higher. Other stringent hybridization conditions may also be selected. Generally, stringent conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Typically, stringent conditions will be those in which the salt concentration is at least about 0.02 molar at pH 7 and the temperature is at least about 50° C. As other factors may significantly affect the stringency of hybridization, including, among others, base composition, length of the nucleic acid strands, the presence of organic solvents, and the extent of base mismatching, the combination of parameters is more important than the absolute measure of any one.

Generally, increased or decreased risk associated with a polymorphism or genetic profile for a disease is indicated by an increased or decreased frequency, respectively, of the disease in a population or individuals harboring the polymorphism or genetic profile, as compared to otherwise similar individuals, who are for instance matched by age, by population, and/or by presence or absence of other polymorphisms associated with risk for the same or similar diseases. The risk effect of a polymorphism can be of different magnitude in different populations. A polymorphism, haplotype, or genetic profile can be negatively associated (“protective polymorphism”) or positively associated (“predisposing polymorphism”) with a complement-related disease such as AMD. The presence of a predisposing genetic profile in an individual can indicate that the individual has an increased risk for the disease relative to an individual with a different profile. Conversely, the presence of a protective polymorphism or genetic profile in an individual can indicate that the individual has a decreased risk for the disease relative to an individual without the polymorphism or profile.

The terms “susceptibility,” “propensity,” and “risk” refer to either an increased or decreased likelihood of an individual developing a disorder (e.g., a condition, illness, disorder or disease) relative to a control and/or non-diseased population. In one example, the control population may be individuals in the population (e.g., matched by age, gender, race and/or ethnicity) without the disorder, or without the genotype or phenotype assayed for.

The terms “diagnose” and “diagnosis” refer to the ability to determine or identify whether an individual has a particular disorder (e.g., a condition, illness, disorder or disease). The term “prognose” or “prognosis” refers to the ability to predict the course of the disease and/or to predict the likely outcome of a particular therapeutic or prophylactic strategy.

The term “screen” or “screening” as used herein has a broad meaning. It includes processes intended for diagnosing or for determining the susceptibility, propensity, risk, or risk assessment of an asymptomatic subject for developing a disorder later in life. Screening also includes the prognosis of a subject, i.e., when a subject has been diagnosed with a disorder, determining in advance the progress of the disorder as well as the assessment of efficacy of therapy options to treat a disorder. Screening can be done by examining a presenting individual's DNA, RNA, or in some cases, protein, to assess the presence or absence of the various SNPs disclosed herein (and typically other SNPs and genetic or behavioral characteristics) so as to determine where the individual lies on the spectrum of disease risk-neutrality-protection. Proxy SNPs may substitute for any of these SNPs. A sample such as a blood sample may be taken from the individual for purposes of conducting the genetic testing using methods known in the art or yet to be developed. Alternatively, if a health provider has access to a pre-produced data set recording all or part of the individual's genome (e.g. a listing of SNPs in the individual's genome), screening may be done simply by inspection of the database, optimally by computerized inspection. Screening may further comprise the step of producing a report identifying the individual and the identity of alleles at the site of at least one or more polymorphisms shown in Table 1 or 2.

### II. Introduction

A study was conducted to elucidate potential associations between complement system genes and other selected genes with age-related macular degeneration (AMD). These genes included, among others, C2 (see, e.g., Bentley (1986) Biochem. J. 239:339-345); Factor B (see, e.g., Woods et al. (1982) PNAS 79 (18): 5661-5 and Mole et al. (1984) J. Biol. Chem. 259 (6): 3407-12); PLEKHA1 (see, e.g., Deloukas et al. (2004) Nature 429 (6990): 375-81; HTRA1/PRSS11 (see, e.g., Zumbrunn et al. (1997) FEBS Lett. 398 (2-3): 187-92 and Zumbrunn et al. (1998) Genomics 45 (2): 461-2); PRELP (see, e.g., Grover et al. (1997) Genomics 38 (2): 109-17); and LOC387715 (see, e.g., International Human Genome Sequencing Consortium (2004) Nature 431 (7011): 931-945). The associations discovered form the basis of the present invention, which provides methods for identifying individuals at increased risk, or at decreased risk, relative to the general population for a complement-related disease such as AMD. The present invention also provides kits, reagents and devices useful for making such determinations. The methods and reagents of the invention are also useful for determining prognosis.

### Use of Polymorphisms to Detect Risk and Protection

The present invention provides a method for detecting an individual's increased or decreased risk for development or progression of a complement-related disease such as AMD by detecting the presence of certain polymorphisms present in the individual's genome that are informative of his or her future disease status (including prognosis and appearance of signs of disease). The presence of such a polymorphism can be regarded as indicative of an individual's risk (increased or decreased) for the disease, especially in individuals who lack other predisposing or protective polymorphisms for the same disease. Even in cases where the predictive contribution of a given polymorphism is relatively minor by itself, genotyping contributes information that nevertheless can be useful in characterizing an individual's predisposition to developing a disease. The information can be particularly useful when combined with genotype information from other loci (e.g., the presence of a certain polymorphism may be more predictive or informative when used in combination with at least one other polymorphism).

### III. New SNPs Associated with Propensity to Develop Disease

In order to identify new single nucleotide polymorphisms (SNPs) associated with increased or decreased risk of developing complement-related diseases such as AMD, 74 complement pathway-associated genes (and a number of inflammation-associated genes including toll-like receptors, or TLRs) were selected for SNP discovery. New SNPs in the candidate genes were discovered from a pool of 475 DNA samples derived from study participants with a history of AMD using a multiplexed SNP enrichment technology called Mismatch Repair Detection (ParAllele Biosciences/Affymetrix), an approach that enriches for variants from pooled samples. This SNP discovery phase (also referred to herein as Phase I) was conducted using DNA derived solely from individuals with AMD based upon the rationale that the discovered SNPs might be highly relevant to disease (e.g., AMD-associated).

### IV. Association of SNPs and Complement-Related Conditions

In Phase II of the study, 1162 DNA samples were employed for genotyping known and newly discovered SNPs in 340 genes. Genes investigated in Phase II included the complement and inflammation-associated genes used for SNP Discovery (Phase I). The remaining genes were selected based upon a tiered strategy, which was designed as follows. Genes received the highest priority if they fell within an AMD-harboring locus established by genome-wide linkage analysis or conventional linkage, or if they were differentially expressed at the RPE-choroid interface in donors with AMD compared to donors without AMD. Particular attention was paid to genes known to participate in inflammation, immune-associated processes, coagulation/fibrinolysis and/or extracellular matrix homeostasis.

In choosing SNPs for these genes, a higher SNP density in the genic regions, which was defined as 5 Kb upstream from the start of transcription until 5 Kb downstream from the end of transcription, was applied. In these regions, an average density of 1 SNP per 10 Kb was used. In the non-genic regions of clusters of complement-related genes, an average of 1 SNP per 20 Kb was employed. The SNPs were chosen from HapMap data in the Caucasian population, the SNP Consortium (Marshall [1999] Science 284[5413]: 406-407), Whitehead, NCBI and the Celera SNP database. Selection included intronic SNPs, variants from the regulatory regions (mainly promoters) and coding SNPs (cSNPs) included in open reading frames. Data obtained by direct screening were used to validate the information extracted from databases. The overall sequence variation of functionally important regions of candidate genes was investigated, not merely a few polymorphisms, using a previously described algorithm for tag selection.

Positive controls included CEPH members (i.e., DNA samples derived from lymphoblastoid cell lines from 61 reference families provided to the NIGMS Repository by the Centre de'Etude du Polymorphism Humain (CEPH), Foundation Jean Dausset in Paris, France) of the HapMap trios; the nomenclature used for these samples is the Coriell sample name (i.e., family relationships were verified by the Coriell Institute for Medical Research Institute for Medical Research). The panel also contained a limited number of X-chromosome probes from two regions. These were included to provide additional information for inferring sample sex. Specifically, if the sample is clearly heterozygous for any X-chromosome markers, it must have two X-chromosomes. However, because there are a limited number of X-chromosome markers in the panel, and because their physical proximity likely means that there are even fewer haplotypes for these markers, we expected that samples with two X-chromosomes might also genotype as homozygous for these markers. The standard procedure for checking sample concordance involved two steps. The first step was to compare all samples with identical names for repeatability. In this study, the only repeats were positive controls and those had repeatability greater than 99.3% (range 99.85% to 100%). The second step was to compare all unique samples to all other unique samples and identify highly concordant sample pairs. Highly concordant sample pairs were used to identify possible tracking errors. The concordance test resulted in 20 sample pairs with concordance greater than 99%.

Samples were genotyped using multiplexed Molecular Inversion Probe (MIP) technology (ParAllele Biosciences/Affymetrix). Successful genotypes were obtained for 3,267 SNPs in 347 genes in 1113 unique samples (out of 1162 unique submitted samples; 3,267 successful assays out 3,308 assays attempted). SNPs with more than 5% failed calls (45 SNPs), SNPs with no allelic variation (354 alleles) and subjects with more than 5% missing genotypes (11 subjects) were deleted.

The resulting genotype data were analyzed in multiple sub-analyses, using a variety of appropriate statistical analyses, as described below.

### A. Polymorphisms Associated with AMD:

One genotype association analysis was performed on all SNPs comparing samples derived from individuals with AMD to those derived from an ethnic- and age-matched control cohort. All genotype associations were assessed using a statistical software program known as SAS®. SNPs showing significant association with AMD are shown in the Tables. Tables 1 and 1A include SNPs from C2, Factor B, PLEKHA1, HTRA1, PRELP, and LOC387715, with additional raw data provided in Tables 2 and 2A as discussed in greater detail hereinbelow. Gene identifiers based on the EnsEMBL database for C2, Factor B, PLEKHA1, HTRA1, and PRELP are provided in Table 5. Table 3 includes SNPs from the RCA locus from FHR1 through F13B. Table 4 includes SNPs from other genes. The genotypes depicted in the Tables are organized alphabetically by gene symbol. AMD associated SNPs identified in a given gene are designated by SNP number or MRD designation. For each SNP, allele frequencies are shown as percentages in both control and disease (AMD) populations. Allele frequencies are provided for individuals homozygous for allele 1 and allele 2, and for heterozygous individuals. For example, for SNP rs1042663, which is located in complement component C2 (C2), 1% of the control population is homozygous for allele 1 (i.e., the individual has an “A” base at this position), 82.1% of the control population is homozygous for allele 2 (i.e., the individual has a “G” base at this position), and 16.9% of the control population is heterozygous. The overall frequency for allele 1 (i.e., the “A” allele) in the control population is 9.5% and the overall frequency for allele 2 in the control population is 90.5%. In the AMD population, 0.4% of the population is homozygous for allele 1 (the “A” allele), 87.9% of population is homozygous for allele 2 (the “G” allele), and 11.7% of the population is heterozygous. The overall frequency for allele 1 (the “A” allele) in the AMD population is 6.2% and the overall frequency for allele 2 (the “G” allele) in the AMD population is 93.8%. Genotype-Likelihood Ratio (3 categories) and Chi Square values (“Freq. Chi Square (both collapsed-2 categories)”) are provided for each SNP. Tables 6 and 6A provide the nucleotide sequences flanking the SNPs disclosed in Tables 1 and 1A. For each sequence, the “N” refers to the polymorphic site. The nucleotide present at the polymorphic site is either allele 1 or allele 2 as shown in Tables 1 and 1A.

In some cases in Tables 3 and 4, “MRD” designations derived from discovered SNPs are provided in place of SNP number designations. MRD_{—}3905 corresponds to the following sequence, which is the region flanking a SNP present in the FHR5 gene:

(SEQ ID NO: 1)
TGCAGAAAAGGATGCGTGTGAACAGCAGGTA(A/G)
TTTTCTTCTGATTGATTCTATATCTAGATGA.

MRD_{—}3906 corresponds to the following sequence, which is the region flanking another SNP present in the FHR5 gene:

(SEQ ID NO: 2)
GGGGAAAAGCAGTGTGGAAATTATTTAGGAC(C/T)GTGTTCATTAATTT
AAAGCAAGGCAAGTCAG.

MRD_{—}4048 corresponds to the following sequence AGCTTCGATATGACTCCACCTGTGAACGTCT(C/G)TACTATGGAGATGATGAGAA ATACTTTCGGA, which is the region flanking the SNP present in the C8A gene: (SEQ ID NO: 3). MRD_{—}4044 corresponds to the following sequence AGGAGAGTAAGACGGGCAGCTACACCCGCAG(A/C)AGTTACCTGCCAGCTGAGC AACTGGTCAGAG, which is the region flanking the SNP present in the C8A gene: (SEQ ID NO: 4). MRD_{—}4452 corresponds to the following sequence GCGTGGTCAGGGGCTGAGTTTTCCAGTTCAG(A/G)ATCAGGACTATGGAGGCACA ACATGGAGGCC, which is the region flanking the SNP present in the CLU gene: (SEQ ID NO: 5). The polymorphic site indicating the SNP associated alleles are shown in parentheses. Further, certain SNPs presented in the Tables were previously identified by MRD designations in U.S. Application No. 60/984,702. For example, in Table 1, rs4151671 is also called MRD_{—}4444. In Table 3, rs1412631 is also called MRD_{—}3922 and rs12027476 is also called MRD_{—}3863. In Table 4, rs2511988 is also called MRD_{—}4083; rs172376 is also called MRD_{—}4035; rs61917913 is also called MRD_{—}4110; rs2230214 is also called MRD_{—}4475; rs10985127 is also called MRD_{—}4477; rs10985126 is also called MRD_{—}4476; rs7857015 is also called MRD_{—}4502; rs3012788 is also called MRD_{—}4495; rs2230429 is also called MRD_{—}4146; rs12142107 is also called MRD_{—}3848; and rs2547438 is also called MRD_{—}4273; rs2230199 is also called MRD_{—}4274; rs1047286 is also called MRD 4270; and rs11085197 is also called MRD_{—}4269.

The presence in the genome or transcripome of an individual of one or more polymorphisms listed in Table 1 is associated with an increased or decreased risk of AMD. Accordingly, detection of a polymorphism shown in Table 1 in a nucleic acid sample of an individual can indicate that the individual is at increased risk for developing AMD. One of skill in the art will be able to refer to Table 1 to identify alleles associated with increased (or decreased) likelihood of developing AMD. For example, in the C2 gene, allele 2 of the SNP rs1042663 is found in 93.8% of AMD chromosomes, but only in 90.5% of the control chromosomes, indicating that a person having allele 2 has a greater likelihood of developing AMD than a person not having allele 2 (See Table 1). The “G” allele is the more common allele (i.e. the “wild type” allele). The “A” allele is the rarer allele, but is more prevalent in the control population than in the AMD population: it is therefore a “protective polymorphism.” Tables 2A and 2B provide the raw data from which the percentages of allele frequencies as shown in Tables 1 and 1A were calculated. Table 2C depicts the absolute values of the differences in frequencies of homozygotes for allele 1 and allele 2 between control and disease populations, the absolute values of the differences in frequencies of heterozygotes between control and disease populations, and the absolute values of the differences in percentages of undetermined subjects between control and disease populations.

In other embodiments, the presence of a combination of multiple (e.g., two or more, three or more, four or more, or five or more) AMD-associated polymorphisms shown in Table 1 and/or 1A indicates an increased (or decreased) risk for AMD.

In addition to the new AMD SNP associations defined herein, these experiments confirmed previously reported associations of AMD with variations/SNPs in the CFH, FHR1-5, F13B, LOC387715, PLEKHA1 and HTRA1 genes.

### V. Determination of Risk (Screening)

### Determining the Risk of an Individual

An individual's relative risk (i.e., susceptibility or propensity) of developing a particular complement-related disease characterized by dysregulation of the complement system can be determined by screening for the presence or absence of a genetic profile that includes one or more single nucleotide polymorphisms (SNPs) selected from Table 1. In a preferred embodiment, the complement-related disease characterized by complement dysregulation is AMD. The presence of any one of the SNPs listed in Table 1 is informative (i.e., indicative) of an individual's risk (increased or decreased) of developing AMD or for predicting the course of progression of AMD in the individual.

The predictive value of a genetic profile for AMD can be increased by screening for a combination of SNPs selected from Table 1 and/or 1A. In one embodiment, the predictive value of a genetic profile is increased by screening for the presence of at least 2 SNPs, at least 3 SNPs, at least 4 SNPs, at least 5 SNPs, at least 6 SNPs, at least 7 SNPs, at least 8 SNPs, at least 9 SNPs, or at least 10 SNPs selected from Table 1 and/or 1A. In another embodiment, the predictive value of a genetic profile for AMD is increased by screening for the presence of at least one SNP from Table 1 and/or 1A and at least one additional SNP selected from the group consisting of a polymorphism in exon 22 of CFH (R1210C), rs1061170, rs203674, rs1061147, rs2274700, rs12097550, rs203674, rs9427661, rs9427662, rs10490924, rs11200638, rs2230199, rs800292, rs3766404, rs529825, rs641153, rs4151667, rs547154, rs9332739, rs3753395, rs1410996, rs393955, rs403846, rs1329421, rs10801554, rs12144939, rs12124794, rs2284664, rs16840422, and rs6695321. In certain embodiments, the method may include screening for at least one SNP from Table 1 and at least one additional SNP associated with risk of AMD selected from the group consisting of: a polymorphism in exon 22 of CFH (R1210C), rs1061170, rs203674, rs1061147, rs2274700, rs12097550, rs203674, rs9427661, rs9427662, rs10490924, rs11200638, and rs2230199.

The predictive value of a genetic profile for AMD can also be increased by screening for a combination of predisposing and protective polymorphisms. For example, the absence of at least one, typically multiple, predisposing polymorphisms and the presence of at least one, typically multiple, protective polymorphisms may indicate that the individual is not at risk of developing AMD. Alternatively, the presence of at least one, typically multiple, predisposing SNPs and the absence of at least one, typically multiple, protective SNPs indicate that the individual is at risk of developing AMD. In one embodiment, a genetic profile for AMD comprises screening for the presence of at least one SNP selected from Table 1 and/or 1A and the presence or absence of at least one protective SNP selected from the group consisting of: rs800292, rs3766404, rs529825, rs641153, rs4151667, rs547154, and rs9332739.

In some embodiments, the genetic profile for AMD includes at least one SNP from C2 and/or Factor B. In one embodiment, the at least one SNP includes rs1042663. In one embodiment, the at least one SNP includes rs4151670. In one embodiment, the at least one SNP includes rs4151650. In one embodiment, the at least one SNP includes rs4151671. In one embodiment, the at least one SNP includes rs4151672. In one embodiment, the at least one SNP includes rs550513.

In some embodiments, the genetic profile for AMD includes at least one SNP from PLEKHAL In one embodiment, the at least one SNP includes rs6585827. In one embodiment, the at least one SNP includes rs10887150. In one embodiment, the at least one SNP includes rs2421018. In one embodiment, the at least one SNP includes rs 10082476. In one embodiment, the at least one SNP includes rs10399971. In one embodiment, the at least one SNP includes rs17649042.

In some embodiments, the genetic profile for AMD includes at least one SNP from HTRA1. In one embodiment, the at least one SNP includes rs4237540. In one embodiment, the at least one SNP includes rs2268345. In one embodiment, the at least one SNP includes re878107. In one embodiment, the at least one SNP includes rs 2253755.

In one embodiment, the genetic profile for AMD includes rs 947367. In one embodiment, the genetic profile for AMD includes rs3750847.

Although the predictive value of the genetic profile can generally be enhanced by the inclusion of multiple SNPs, no one of the SNPs is indispensable. Accordingly, in various embodiments, one or more of the SNPs is omitted from the genetic profile.

In certain embodiments, the genetic profile comprises a combination of at least two SNPs selected from the pairs identified below:

Exemplary pairwise combinations of informative SNPs

| | rs104 | rs415 | rs415 | rs415 | rs415 | rs55 | rs658 | rs108 | rs242 | rs100 | rs103 | rs176 | rs423 | rs226 | rs87 | rs94 | rs375 | rs225 |
|---|---|---|---|---|---|---|---|---|---|---|---|---|---|---|---|---|---|---|
| | 2663 | 1670 | 1650 | 1671 | 1672 | 0513 | 5827 | 87150 | 1018 | 82476 | 99971 | 49042 | 7540 | 8345 | 8107 | 7367 | 0847 | 3755 |
| rs1042663 | | X | X | X | X | X | X | X | X | X | X | X | X | X | X | X | X | X |
| rs4151670 | X | | X | X | X | X | X | X | X | X | X | X | X | X | X | X | X | X |
| rs4151650 | X | X | | X | X | X | X | X | X | X | X | X | X | X | X | X | X | X |
| rs4151671 | X | X | X | | X | X | X | X | X | X | X | X | X | X | X | X | X | X |
| rs4151672 | X | X | X | X | | X | X | X | X | X | X | X | X | X | X | X | X | X |
| rs550513 | X | X | X | X | X | | X | X | X | X | X | X | X | X | X | X | X | X |
| rs6585827 | X | X | X | X | X | X | | X | X | X | X | X | X | X | X | X | X | X |
| rs10887150 | X | X | X | X | X | X | X | | X | X | X | X | X | X | X | X | X | X |
| rs2421018 | X | X | X | X | X | X | X | X | | X | X | X | X | X | X | X | X | X |
| rs10082476 | X | X | X | X | X | X | X | X | X | | X | X | X | X | X | X | X | X |
| rs10399971 | X | X | X | X | X | X | X | X | X | X | | X | X | X | X | X | X | X |
| rs17649042 | X | X | X | X | X | X | X | X | X | X | X | | X | X | X | X | X | X |
| rs4237540 | X | X | X | X | X | X | X | X | X | X | X | X | | X | X | X | X | X |
| rs2268345 | X | X | X | X | X | X | X | X | X | X | X | X | X | | X | X | X | X |
| rs878107 | X | X | X | X | X | X | X | X | X | X | X | X | X | X | | X | X | X |
| rs947367 | X | X | X | X | X | X | X | X | X | X | X | X | X | X | X | | X | X |
| rs3750847 | X | X | X | X | X | X | X | X | X | X | X | X | X | X | X | X | | X |
| rs2253755 | X | X | X | X | X | X | X | X | X | X | X | X | X | X | X | X | X | |

In a further embodiment, the determination of an individual's genetic profile can also include screening for a deletion or a heterozygous deletion that is associated with AMD risk or protection. Exemplary deletions that are associated with AMD protection include a deletion in FHR3 and FHR1 genes. The deletion may encompass one gene, multiple genes, a portion of a gene, or an intergenic region, for example. If the deletion impacts the size, conformation, expression or stability of an encoded protein, the deletion can be detected by assaying the protein, or by querying the nucleic acid sequence of the genome or transcriptome of the individual.

Further, determining an individual's genetic profile may include determining an individual's genotype or haplotype to determine if the individual is at an increased or decreased risk of developing AMD. In one embodiment, an individual's genetic profile may comprise SNPs that are in linkage disequilibrium with other SNPs associated with AMD that define a haplotype (e.g., a set of polymorphisms on chromosome 10 in or near PLEKHA1, LOC387715, and HTRA1) associated with risk or protection of AMD. In another embodiment, a genetic profile may include multiple haplotypes present in the genome or a combination of haplotypes and polymorphisms, such as single nucleotide polymorphisms, in the genome, e.g., a haplotype on chromosome 10 and a haplotype or at least one SNP on chromosome 6.

Further studies of the identity of the various SNPs and other genetic characteristics disclosed herein with additional cohorts, and clinical experience with the practice of this invention on populations, will permit ever more precise assessment of AMD risk based on emergent SNP patterns. This work will result in refinement of which particular set of SNPs are characteristic of a genetic profile which is, for example, indicative of an urgent need for intervention, or indicative that the early stage of AMD observed in an individual is unlikely to progress to more serious disease, or is likely to progress rapidly to the wet form of the disease, or that the presenting individual is not at significant risk of developing AMD, or that a particular AMD therapy is most likely to be successful with this individual and another therapeutic alternative less likely to be productive. Thus, it is anticipated that the practice of the invention disclosed herein, especially when combined with the practice of risk assessment using other known risk-indicative and protection-indicative SNPs, will permit disease management and avoidance with increasing precision.

A single nucleotide polymorphism within a genetic profile for AMD as described herein may be detected directly or indirectly. Direct detection refers to determining the presence or absence of a specific SNP identified in the genetic profile using a suitable nucleic acid, such as an oligonucleotide in the form of a probe or primer as described below. Alternatively, direct detection can include querying a pre-produced database comprising all or part of the individual's genome for a specific SNP in the genetic profile. Other direct methods are known to those skilled in the art. Indirect detection refers to determining the presence or absence of a specific SNP identified in the genetic profile by detecting a surrogate or proxy SNP that is in linkage disequilibrium with the SNP in the individual's genetic profile. Detection of a proxy SNP is indicative of a SNP of interest and is increasingly informative to the extent that the SNPs are in linkage disequilibrium, e.g., at least 50%, 60%, 70%, 80%, 90%, 95%, 98%, or about 100% LD. Another indirect method involves detecting allelic variants of proteins accessible in a sample from an individual that are consequent of a risk-associated or protection-associated allele in DNA that alters a codon.

It is also understood that a genetic profile as described herein may include one or more nucleotide polymorphism(s) that are in linkage disequilibrium with a polymorphism that is causative of disease. In this case, the SNP in the genetic profile is a surrogate SNP for the causative polymorphism.

Genetically linked SNPs, including surrogate or proxy SNPs, can be identified by methods known in the art. Non-random associations between polymorphisms (including single nucleotide polymorphisms, or SNPs) at two or more loci are measured by the degree of linkage disequilibrium (LD). The degree of linkage disequilibrium is influenced by a number of factors including genetic linkage, the rate of recombination, the rate of mutation, random drift, non-random mating and population structure. Moreover, loci that are in LD do not have to be located on the same chromosome, although most typically they occur as clusters of adjacent variations within a restricted segment of DNA. Polymorphisms that are in complete or close LD with a particular disease-associated SNP are also useful for screening, diagnosis, and the like.

SNPs in LD with each other can be identified using methods known in the art and SNP databases (e.g., the Perlegen database, at http://genome.perlegen.com/browser/download.html and others). For illustration, SNPs in linkage disequilibrium (LD) with the CFH SNP rs800292 were identified using the Perlegen database. This database groups SNPs into LD bins such that all SNPs in the bin are highly correlated to each other. For example, AMD-associated SNP rs800292 was identified in the Perlegen database under the identifier ‘afd0678310’. A LD bin (European LD bin #1003371; see table below) was then identified that contained linked SNPs—including afd1152252, afd4609785, afd4270948, afd0678315, afd0678311, and afd0678310—and annotations.

| | | | | | | Allele |
|---|---|---|---|---|---|---|
| SNP ID | | | | | | Frequency |
| Perlegen | | SNP Position | | | | European |
| ‘afd’ ID* | ‘ss’ ID | Chromosome | Accession | Position | Alleles | American |
| afd1152252 | ss23875287 | 1 | NC_000001.5 | 193872580 | A/G | 0.21 |
| afd4609785 | ss23849009 | 1 | NC_000001.5 | 193903455 | G/A | 0.79 |
| afd4270948 | ss23849019 | 1 | NC_000001.5 | 193905168 | T/C | 0.79 |
| afd0678315 | ss23857746 | 1 | NC_000001.5 | 193923365 | G/A | 0.79 |
| afd0678311 | ss23857767 | 1 | NC_000001.5 | 193930331 | C/T | 0.79 |
| afd0678310 | ss23857774 | 1 | NC_000001.5 | 193930492 | G/A | 0.79 |

*Perlegen AFD identification numbers can be converted into conventional SNP database identifiers (in this case, rs4657825, rs576258, rs481595, rs529825, rs551397, and rs800292) using the NCBI database (http://www.ncbi.nlm.nih.gov/sites/entrez?db=snp&cmd=search&term=).

The frequencies of these alleles in disease versus control populations may be determined using the methods described herein.

As a second example, the LD tables computed by HapMap were downloaded (http://ftp.hapmap.org/ld_data/latest/). Unlike the Perlegen database, the HapMap tables use ‘rs’ SNP identifiers directly. All SNPs with an R² value greater than 0.80 when compared to rs800292 were extracted from the database in this illustration. Due to the alternate threshold used to compare SNPs and the greater SNP coverage of the HapMap data, more SNPs were identified using the HapMap data than the Perlegen data.

| SNP 1 | SNP #2 | | SNP | | | | |
|---|---|---|---|---|---|---|---|
| Location | Location | Population | #1 ID | SNP #2 ID | D′ | R² | LOD |
| 194846662 | 194908856 | CEU | rs10801551 | rs800292 | 1 | 0.84 | 19.31 |
| 194850944 | 194908856 | CEU | rs4657825 | rs800292 | 1 | 0.9 | 21.22 |
| 194851091 | 194908856 | CEU | rs12061508 | rs800292 | 1 | 0.83 | 18.15 |
| 194886125 | 194908856 | CEU | rs505102 | rs800292 | 1 | 0.95 | 23.04 |
| 194899093 | 194908856 | CEU | rs6680396 | rs800292 | 1 | 0.84 | 19.61 |
| 194901729 | 194908856 | CEU | rs529825 | rs800292 | 1 | 0.95 | 23.04 |
| 194908856 | 194928161 | CEU | rs800292 | rs12124794 | 1 | 0.84 | 18.81 |
| 194908856 | 194947437 | CEU | rs800292 | rs1831281 | 1 | 0.84 | 19.61 |
| 194908856 | 194969148 | CEU | rs800292 | rs2284664 | 1 | 0.84 | 19.61 |
| 194908856 | 194981223 | CEU | rs800292 | rs10801560 | 1 | 0.84 | 19.61 |
| 194908856 | 194981293 | CEU | rs800292 | rs10801561 | 1 | 0.84 | 19.61 |
| 194908856 | 195089923 | CEU | rs800292 | rs10922144 | 1 | 0.84 | 19.61 |

As indicated above, publicly available databases such as the HapMap database (http://ftp.hapmap.org/ld_data/latest/) and Haploview (Barrett, J. C. et al., Bioinformatics 21, 263 (2005)) may be used to calculate linkage disequilibiurm between two SNPs. The frequency of identified alleles in disease versus control populations may be determined using the methods described herein. Statistical analyses may be employed to determine the significance of a non-random association between the two SNPs (e.g., Hardy-Weinberg Equilibrium, Genotype likelihood ratio (genotype p value), Chi Square analysis, Fishers Exact test). A statistically significant non-random association between the two SNPs indicates that they are in linkage disequilibrium and that one SNP can serve as a proxy for the second SNP.

The screening step to determine an individual's genetic profile may be conducted by inspecting a data set indicative of genetic characteristics previously derived from analysis of the individual's genome. A data set indicative of an individual's genetic characteristics may include a complete or partial sequence of the individual's genomic DNA, or a SNP map. Inspection of the data set including all or part of the individual's genome may optimally be performed by computer inspection. Screening may further comprise the step of producing a report identifying the individual and the identity of alleles at the site of at least one or more polymorphisms shown in Table 1 or 1A and/or proxy SNPs.

Alternatively, the screening step to determine an individual's genetic profile includes analyzing a nucleic acid (i.e., DNA or RNA) sample obtained from the individual. A sample can be from any source containing nucleic acids (e.g., DNA or RNA) including tissues such as hair, skin, blood, biopsies of the retina, kidney, or liver or other organs or tissues, or sources such as saliva, cheek scrapings, urine, amniotic fluid or CVS samples, and the like. Typically, genomic DNA is analyzed. Alternatively, RNA, cDNA, or protein can be analyzed. Methods for the purification or partial purification of nucleic acids or proteins from a sample, and various protocols for analyzing samples for use in diagnostic assays are well known.

A polymorphism such as a SNP can be conveniently detected using suitable nucleic acids, such as oligonucleotides in the form of primers or probes. Accordingly, the invention not only provides novel SNPs and/or novel combinations of SNPs that are useful in assessing risk for a complement-related disease, but also nucleic acids such as oligonucleotides useful to detect them. A useful oligonucleotide for instance comprises a sequence that hybridizes under stringent hybridization conditions to at least one polymorphism identified herein. Where appropriate, at least one oligonucleotide includes a sequence that is fully complementary to a nucleic acid sequence comprising at least one polymorphism identified herein. Such oligonucleotide(s) can be used to detect the presence of the corresponding polymorphism, for example by hybridizing to the polymorphism under stringent hybridizing conditions, or by acting as an extension primer in either an amplification reaction such as PCR or a sequencing reaction, wherein the corresponding polymorphism is detected either by amplification or sequencing. Suitable detection methods are described below.

An individual's genotype can be determined using any method capable of identifying nucleotide variation, for instance at single nucleotide polymorphic sites. The particular method used is not a critical aspect of the invention. Although considerations of performance, cost, and convenience will make particular methods more desirable than others, it will be clear that any method that can detect one or more polymorphisms of interest can be used to practice the invention. A number of suitable methods are described below.

### 1) Nucleic Acid Analysis

### General

Polymorphisms can be identified through the analysis of the nucleic acid sequence present at one or more of the polymorphic sites. A number of such methods are known in the art. Some such methods can involve hybridization, for instance with probes (probe-based methods). Other methods can involve amplification of nucleic acid (amplification-based methods). Still other methods can include both hybridization and amplification, or neither.

### a) Amplification-Based Methods

### Preamplification Followed by Sequence Analysis:

Where useful, an amplification product that encompasses a locus of interest can be generated from a nucleic acid sample. The specific polymorphism present at the locus is then determined by further analysis of the amplification product, for instance by methods described below. Allele-independent amplification can be achieved using primers which hybridize to conserved regions of the genes. The genes contain many invariant or monomorphic regions and suitable allele-independent primers can be selected routinely.

Upon generation of an amplified product, polymorphisms of interest can be identified by DNA sequencing methods, such as the chain termination method (Sanger et al., 1977, Proc. Natl. Acad. Sci,. 74:5463-5467) or PCR-based sequencing. Other useful analytical techniques that can detect the presence of a polymorphism in the amplified product include single-strand conformation polymorphism (SSCP) analysis, denaturing gradient gel electropohoresis (DGGE) analysis, and/or denaturing high performance liquid chromatography (DHPLC) analysis. In such techniques, different alleles can be identified based on sequence- and structure-dependent electrophoretic migration of single stranded PCR products. Amplified PCR products can be generated according to standard protocols, and heated or otherwise denatured to form single stranded products, which may refold or form secondary structures that are partially dependent on base sequence. An alternative method, referred to herein as a kinetic-PCR method, in which the generation of amplified nucleic acid is detected by monitoring the increase in the total amount of double-stranded DNA in the reaction mixture, is described in Higuchi et al., 1992, Bio/Technology, 10:413-417, incorporated herein by reference.

### Allele-Specific Amplification:

Alleles can also be identified using amplification-based methods. Various nucleic acid amplification methods known in the art can be used in to detect nucleotide changes in a target nucleic acid. Alleles can also be identified using allele-specific amplification or primer extension methods, in which amplification or extension primers and/or conditions are selected that generate a product only if a polymorphism of interest is present.

### Amplification Technologies

A preferred method is the polymerase chain reaction (PCR), which is now well known in the art, and described in U.S. Pat. Nos. 4,683,195; 4,683,202; and 4,965,188; each incorporated herein by reference. Other suitable amplification methods include the ligase chain reaction (Wu and Wallace, 1988, Genomics 4:560-569); the strand displacement assay (Walker et al., 1992, Proc. Natl. Acad. Sci. USA 89:392-396, Walker et al. 1992, Nucleic Acids Res. 20:1691-1696, and U.S. Pat. No. 5,455,166); and several transcription-based amplification systems, including the methods described in U.S. Pat. Nos. 5,437,990; 5,409,818; and 5,399,491; the transcription amplification system (TAS) (Kwoh et al., 1989, Proc. Natl. Acad. Sci. USA, 86:1173-1177); and self-sustained sequence replication (3SR) (Guatelli et al., 1990, Proc. Natl. Acad. Sci. USA, 87:1874-1878 and WO 92/08800); each incorporated herein by reference. Alternatively, methods that amplify the probe to detectable levels can be used, such as QB-replicase amplification (Kramer et al., 1989, Nature, 339:401-402, and Lomeli et al., 1989, Clin. Chem., 35:1826-1831, both of which are incorporated herein by reference). A review of known amplification methods is provided in Abramson et al., 1993, Current Opinion in Biotechnology, 4:41-47, incorporated herein by reference.

Amplification of mRNA

Genotyping also can also be carried out by detecting and analyzing mRNA under conditions when both maternal and paternal chromosomes are transcribed. Amplification of RNA can be carried out by first reverse-transcribing the target RNA using, for example, a viral reverse transcriptase, and then amplifying the resulting cDNA, or using a combined high-temperature reverse-transcription-polymerase chain reaction (RT-PCR), as described in U.S. Pat. Nos. 5,310,652; 5,322,770; 5,561,058; 5,641,864; and 5,693,517; each incorporated herein by reference (see also Myers and Sigua, 1995, in PCR Strategies, supra, chapter 5).

### Selection of Allele-Specific Primers

The design of an allele-specific primer can utilize the inhibitory effect of a terminal primer mismatch on the ability of a DNA polymerase to extend the primer. To detect an allele sequence using an allele-specific amplification or extension-based method, a primer complementary to the genes of interest is chosen such that the nucleotide hybridizes at or near the polymorphic position. For instance, the primer can be designed to exactly match the polymorphism at the 3′ terminus such that the primer can only be extended efficiently under stringent hybridization conditions in the presence of nucleic acid that contains the polymorphism. Allele-specific amplification- or extension-based methods are described in, for example, U.S. Pat. Nos. 5,137,806; 5,595,890; 5,639,611; and U.S. Pat. No. 4,851,331, each incorporated herein by reference.

### Analysis of Heterozygous Samples

If so desired, allele-specific amplification can be used to amplify a region encompassing multiple polymorphic sites from only one of the two alleles in a heterozygous sample.

### b) Probe-Based Methods

### General

Alleles can be also identified using probe-based methods, which rely on the difference in stability of hybridization duplexes formed between a probe and its corresponding target sequence comprising an allele. For example, differential probes can be designed such that under sufficiently stringent hybridization conditions, stable duplexes are formed only between the probe and its target allele sequence, but not between the probe and other allele sequences.

Probe design

A suitable probe for instance contains a hybridizing region that is either substantially complementary or exactly complementary to a target region of a polymorphism described herein or their complement, wherein the target region encompasses the polymorphic site. The probe is typically exactly complementary to one of the two allele sequences at the polymorphic site. Suitable probes and/or hybridization conditions, which depend on the exact size and sequence of the probe, can be selected using the guidance provided herein and well known in the art. The use of oligonucleotide probes to detect nucleotide variations including single base pair differences in sequence is described in, for example, Conner et al., 1983, Proc. Natl. Acad. Sci. USA, 80:278-282, and U.S. Pat. Nos. 5,468,613 and 5,604,099, each incorporated herein by reference.

### Pre-Amplification Before Probe Hybridization

In an embodiment, at least one nucleic acid sequence encompassing one or more polymorphic sites of interest are amplified or extended, and the amplified or extended product is hybridized to one or more probes under sufficiently stringent hybridization conditions. The alleles present are inferred from the pattern of binding of the probes to the amplified target sequences.

### Some Known Probe-Based Genotyping Assays

Probe-based genotyping can be carried out using a “TaqMan” or “5′-nuclease assay,” as described in U.S. Pat. Nos. 5,210,015; 5,487,972; and 5,804,375; and Holland et al., 1988, Proc. Natl. Acad. Sci. USA, 88:7276-7280, each incorporated herein by reference. Examples of other techniques that can be used for SNP genotyping include, but are not limited to, Amplifluor, Dye Binding-Intercalation, Fluorescence Resonance Energy Transfer (FRET), Hybridization Signal Amplification Method (HSAM), HYB Probes, Invader/Cleavase Technology (Invader/CFLP), Molecular Beacons, Origen, DNA-Based Ramification Amplification (RAM), rolling circle amplification, Scorpions, Strand displacement amplification (SDA), oligonucleotide ligation (Nickerson et al., Proc. Natl Acad. Sci. USA, 87: 8923-8927) and/or enzymatic cleavage. Popular high-throughput SNP-detection methods also include template-directed dye-terminator incorporation (TDI) assay (Chen and Kwok, 1997, Nucleic Acids Res. 25: 347-353), the 5′-nuclease allele-specific hybridization TaqMan assay (Livak et al. 1995, Nature Genet. 9: 341-342), and the recently described allele-specific molecular beacon assay (Tyagi et al. 1998, Nature Biotech. 16: 49-53).

### Assay Formats

Suitable assay formats for detecting hybrids formed between probes and target nucleic acid sequences in a sample are known in the art and include the immobilized target (dot-blot) format and immobilized probe (reverse dot-blot or line-blot) assay formats. Dot blot and reverse dot blot assay formats are described in U.S. Pat. Nos. 5,310,893; 5,451,512; 5,468,613; and 5,604,099; each incorporated herein by reference. In some embodiments multiple assays are conducted using a microfluidic format. See, e.g., Unger et al., 2000, Science 288:113-6.

### Nucleic Acids Containing Polymorphisms of Interest

The invention also provides isolated nucleic acid molecules, e.g., oligonucleotides, probes and primers, comprising a portion of the genes, their complements, or variants thereof as identified herein. Preferably the variant comprises or flanks at least one of the polymorphic sites identified herein, such as variants associated with AMD.

Nucleic acids such as primers or probes can be labeled to facilitate detection. Oligonucleotides can be labeled by incorporating a label detectable by spectroscopic, photochemical, biochemical, immunochemical, radiological, radiochemical or chemical means. Useful labels include ³²P, fluorescent dyes, electron-dense reagents, enzymes, biotin, or haptens and proteins for which antisera or monoclonal antibodies are available.

### 2) Protein-Based or Phenotypic Detection of Polymorphism

Where polymorphisms are associated with a particular phenotype, then individuals that contain the polymorphism can be identified by checking for the associated phenotype. For example, where a polymorphism causes an alteration in the structure, sequence, expression and/or amount of a protein or gene product, and/or size of a protein or gene product, the polymorphism can be detected by protein-based assay methods.

### Techniques for Protein Analysis

Protein-based assay methods include electrophoresis (including capillary electrophoresis and one- and two-dimensional electrophoresis), chromatographic methods such as high performance liquid chromatography (HPLC), thin layer chromatography (TLC), hyperdiffusion chromatography, and mass spectrometry.

### Antibodies

Where the structure and/or sequence of a protein is changed by a polymorphism of interest, one or more antibodies that selectively bind to the altered form of the protein can be used. Such antibodies can be generated and employed in detection assays such as fluid or gel precipitin reactions, immunodiffusion (single or double), immunoelectrophoresis, radioimmnunoassay (RIA), enzyme-linked immunosorbent assays (ELISAs), immunofluorescent assays, Western blotting and others.

### 3) Kits

In certain embodiments, one or more oligonucleotides of the invention are provided in a kit or on an array useful for detecting the presence of a predisposing or a protective polymorphism in a nucleic acid sample of an individual whose risk for a complement-related disease such as AMD is being assessed. A useful kit can contain oligonucleotide specific for particular alleles of interest as well as instructions for their use to determine risk for a complement-related disease such as AMD. In some cases, the oligonucleotides may be in a form suitable for use as a probe, for example fixed to an appropriate support membrane. In other cases, the oligonucleotides can be intended for use as amplification primers for amplifying regions of the loci encompassing the polymorphic sites, as such primers are useful in the preferred embodiment of the invention. Alternatively, useful kits can contain a set of primers comprising an allele-specific primer for the specific amplification of alleles. As yet another alternative, a useful kit can contain antibodies to a protein that is altered in expression levels, structure and/or sequence when a polymorphism of interest is present within an individual. Other optional components of the kits include additional reagents used in the genotyping methods as described herein. For example, a kit additionally can contain amplification or sequencing primers which can, but need not, be sequence-specific, enzymes, substrate nucleotides, reagents for labeling and/or detecting nucleic acid and/or appropriate buffers for amplification or hybridization reactions.

### 4) Arrays

The present invention also relates to an array, a support with immobilized oligonucleotides useful for practicing the present method. A useful array can contain oligonucleotide probes specific for polymorphisms identified herein. The oligonucleotides can be immobilized on a substrate, e.g., a membrane or glass. The oligonucleotides can, but need not, be labeled. The array can comprise one or more oligonucleotides used to detect the presence of one or more SNPs provided herein. In some embodiments, the array can be a micro-array.

The array can include primers or probes to determine assay the presence or absence of at least two of the SNPs listed in Table 1 and/or 1A, sometimes at least three, at least four, at least five or at least six of the SNPs. In one embodiment, the array comprises probes or primers for detection of fewer than about 1000 different SNPs, often fewer than about 100 different SNPs, and sometimes fewer than about 50 different SNPs.

### VI. Therapeutic Methods

The invention also provides a method for treating or preventing AMD that includes prophylactically or therapeutically treating an individual identified as having a genetic profile characterized by polymorphisms in the genome of the individual indicative of risk for developing AMD, wherein the genetic profile includes one or more single nucleotide polymorphisms selected from Table 1 and/or Table 1A.

An individual with a genetic profile indicative of elevated risk of AMD can be treated by administering a composition comprising a human Complement Factor H polypeptide to the individual. In one embodiment, the Factor H polypeptide is encoded by a Factor H protective haplotype. A protective Factor H haplotype can encode an isoleucine residue at amino acid position 62 and/or an amino acid other than a histidine at amino acid position 402. For example, a Factor H polypeptide can comprise an isoleucine residue at amino acid position 62, a tyrosine residue at amino acid position 402, and/or an arginine residue at amino acid position 1210. Exemplary Factor H protective haplotypes include the H2 haplotype or the H4 haplotype (see U.S. Patent Publication 2007/0020647, which is incorporated by reference in its entirety herein). Alternatively, the Factor H polypeptide may be encoded by a Factor H neutral haplotype. A neutral haplotype encodes an amino acid other than an isoleucine at amino acid position 62 and an amino acid other than a histidine at amino acid position 402. Exemplary Factor H neutral haplotypes include the H3 haplotype or the H5 haplotype (see U.S. Patent Publication 2007/0020647).

A therapeutic Factor H polypeptide may be a recombinant protein or it may be purified from blood. A Factor H polypeptide may be administered to the eye by intraocular injection or systemically.

Alternatively, or in addition, an individual with a genetic profile indicative of elevated risk of AMD could be treated by inhibiting the expression or activity of HTRA1. As one example, HTRA1 can be inhibited by administering an antibody or other protein (e.g. an antibody variable domain, an addressable fibronectin protein, etc.) that binds HTRA1. Alternatively, HTRA1 can be inhibited by administering a small molecule that interferes with HTRA1 activity (e.g. an inhibitor of the protease activity of HTRA1) or a nucleic acid inhibiting HTRA1 expression or activity, such as an inhibitory RNA (e.g. a short interfering RNA, a short hairpin RNA, or a microRNA), a nucleic acid encoding an inhibitory RNA, an antisense nucleic acid, or an aptamer that binds HTRA1. See, for example, International Publication No. WO 2008/013893. An inhibitor for HTRA1 activity, NVP-LBG976, is available from Novartis, Basel (see also, Grau S, PNAS, (2005) 102: 6021-6026). Antibodies reactive to HTRA1 are commercially available (for example from Imgenex) and are also described in, for example, PCT application No. WO 00/08134.

Alternatively, or in addition, the method of treating or preventing AMD in an individual includes prophylactically or therapeutically treating the individual by inhibiting Factor B and/or C2 in the individual. Factor B can be inhibited, for example, by administering an antibody or other protein (e.g., an antibody variable domain, an addressable fibronectin protein, etc.) that binds Factor B. Alternatively, Factor B can be inhibited by administering a nucleic acid inhibiting Factor B expression or activity, such as an inhibitory RNA, a nucleic acid encoding an inhibitory RNA, an antisense nucleic acid, or an aptamer, or by administering a small molecule that interferes with Factor B activity (e.g., an inhibitor of the protease activity of Factor B). C2 can be inhibited, for example, by administering an antibody or other protein (e.g., an antibody variable domain, an addressable fibronectin protein, etc.) that binds C2. Alternatively, C2 can be inhibited by administering a nucleic acid inhibiting C2 expression or activity, such as an inhibitory RNA, a nucleic acid encoding an inhibitory RNA, an antisense nucleic acid, or an aptamer, or by administering a small molecule that interferes with C2 activity (e.g., an inhibitor of the protease activity of C2).

In another embodiment, an individual with a genetic profile indicative of AMD (i.e., the individual's genetic profile comprises one or more single nucleotide polymorphisms selected from Table 1 or Table 1A) can be treated by administering a composition comprising a C3 convertase inhibitor, e.g., compstatin (See e.g. PCT publication WO 2007/076437). optionally in combination with a therapeutic factor H polypeptide and/or an HTRA1 inhibitor. In another embodiment, an individual with a genetic profile indicative of AMD and who is diagnosed with AMD may be treated with an angiogenic inhibitor such as anecortave acetate (RETAANE®, Alcon), an anti-VEGF inhibitor such as pegaptanib (Macugen®, Eyetech Pharmaceuticals and Pfizer, Inc.) and ranibizumab (Lucentis®, Genentech), and/or verteporfin (Visudyne®, QLT, Inc./Novartis).

### VII. Authorization of Treatment or Payment for Treatment

The invention also provides a healthcare method comprising paying for, authorizing payment for or authorizing the practice of the method of screening for susceptibility to developing or for predicting the course of progression of AMD in an individual, comprising screening for the presence or absence of genetic profile characterized by polymorphisms in the genome of the individual indicative of risk for developing AMD, wherein the genetic profile includes one or more single nucleotide polymorphisms selected from Table 1 and/or Table 1A.

According to the methods of the present invention, a third party, e.g., a hospital, clinic, a government entity, reimbursing party, insurance company (e.g., a health insurance company), HMO, third-party payor, or other entity which pays for, or reimburses medical expenses may authorize treatment, authorize payment for treatment, or authorize reimbursement of the costs of treatment. For example, the present invention relates to a healthcare method that includes authorizing the administration of, or authorizing payment or reimbursement for the administration of, a diagnostic assay for determining an individual's susceptibility for developing or for predicting the course of progression of AMD as disclosed herein. For example, the healthcare method can include authorizing the administration of, or authorizing payment or reimbursement for the administration of, a diagnostic assay to determine an individual's susceptibility for development or progression of AMD that includes screening for the presence or absence of a genetic profile that includes one or more SNPs selected from Table 1 and/or 1A.

### VIII. Complement-Related Diseases

The polymorphisms provided herein have a statistically significant association with one or more disorders that involve dysfunction of the complement system. In certain embodiments, an individual may have a genetic predisposition based on their genetic profile to developing more than one disorder associated with dysregulation of the complement system. For example, said individual's genetic profile may comprise one or more polymorphism shown in Table 1 or 1A, wherein the genetic profile is informative of AMD and another disease or condition characterized by dysregulation of the complement system. Accordingly, the invention contemplates the use of these polymorphisms for assessing an individual's risk for any complement-related disease or condition, including but not limited to AMD. Other complement-related diseases include MPGN II, Barraquer-Simons Syndrome, asthma, lupus erythematosus, glomerulonephritis, various forms of arthritis including rheumatoid arthritis, autoimmune heart disease, multiple sclerosis, inflammatory bowel disease, Celiac disease, diabetes mellitus type 1, Sjögren's syndrome, and ischemia-reperfusion injuries. The complement system is also becoming increasingly implicated in diseases of the central nervous system such as Alzheimer's disease, and other neurodegenerative conditions. Applicant suspects that many patients may die of disease caused in part by dysfunction of the complement cascade well before any symptoms of AMD appear. Accordingly, the invention disclosed herein may well be found to be useful in early diagnosis and risk assessment of other disease, enabling opportunistic therapeutic or prophylactic intervention delaying the onset or development of symptoms of such disease.

The examples of the present invention presented below are provided only for illustrative purposes and not to limit the scope of the invention. Numerous embodiments of the invention within the scope of the claims that follow the examples will be apparent to those of ordinary skill in the art from reading the foregoing text and following examples.

### EXAMPLES

Additional sub-analyses were performed to support data derived from analyses described above in Tables 1-4. These include:

Sub-analysis 1: One preliminary sub-analysis was performed on a subset of 2,876 SNPs using samples from 590 AMD cases and 375 controls. It was determined that this sample provided adequate power (>80%) for detecting an association between the selected markers and AMD (for a relative risk of 1.7, a sample size of 500 per group was required, and for a relative risk of 1.5, the sample size was calculated to be 700 per group).

The raw data were prepared for analysis in the following manner: 1) SNPs with more than 5% failed calls were deleted (45 total SNPs); 2) SNPs with no allelic variation were deleted (354 alleles); 3) subjects with more than 5% missing genotypes were deleted (11 subjects); and 4) the 2,876 remaining SNPs were assessed for LD, and only one SNP was retained for each pair with r2 >0.90 (631 SNPs dropped, leaving 2245 SNPs for analysis). Genotype associations were assessed using a statistical software program (i.e., SAS® PROC CASECONTROL) and the results were sorted both by genotype p-value and by allelic p-value. For 2,245 SNPs, the Bonferroni-corrected alpha level for significance is 0.00002227. Seventeen markers passed this test. HWE was assessed for each of the 17 selected markers, both with all data combined and by group.

AMD-associated SNPs were further analyzed to determine q-values. Of 2245 SNPs analyzed, 74 SNPs were shown to be associated with AMD at a q-value less than 0.50. 16 AMD-associated SNPs, located in the CFH, LOC387715, FHR4, FHR5, HTRA1, PLEKHA1 and FHR2 genes passed the Bonferroni level of adjustment. These results confirm the published associations of the CFH and LOC387715, PLEKHAI and HTRA1 genes with AMD. 14 additional SNPs located within the FHR5, FHR2, CFH, HTRA1, FHR1, SPOCK3, PLEKHA1, C2, FBN2, TLR3 and SPOCK loci were significantly associated with AMD; these SNPs didn't pass the Bonferroni cut-off, but had q-values less than 0.20 (after adjusting for false discovery rate). In addition, another 27 SNPs were significantly associated with AMD (p<0.05) at q-values between 0.20 and 0.50.

These data confirm existing gene associations in the literature. They also provide evidence that other complement-associated genes (e.g., FHR1, FHR2, FHR4, FHR5) may not be in linkage disequilibrium (LD) with CFH and, if replicated in additional cohorts, may be independently associated with AMD. It is also noted that FHR1, FHR2 and FHR4 are in the same LD bin and further genotyping will be required to identify the gene(s) within this group that drive the detected association with AMD.

Sub-analysis 2: Another sub-analysis was performed on a subset comprised of 516 AMD cases and 298 controls using criteria as described above. A total of 3,266 SNPs in 352 genes from these regions were tested. High significance was detected for previously established AMD-associated genes, as well as for several novel AMD genes. SNPs exhibiting p values <0.01 and difference in allele frequencies >5% are depicted in Table 1.

Sub-analysis 3: Another sub-analysis was performed comparing 499 AMD cases to 293 controls: data were assessed for Hardy-Weinberg association and analyzed by Chi Square. Using a cutoff of p<0.005, 40 SNPs were significantly associated with AMD; these included SNPs within genes shown previously to be associated with AMD (CFH/ENSG00000000971, CFHR1, CFHR2, CFHR4, CFHR5, F13B, PLEKHA1, LOC387715 and PRSS11/HTRA1), as well as additional strong associations with CCL28 and ADAM12. The same samples were analyzed also by conditioning on the CFH Y402H SNP to determine how much association remained after accounting for this strongly associated SNP using a Cochran-Armitage Chi Square test for association within a bin and a Mantel-Haenszel test for comparing bins. The significance of association for most markers in the CFH region drops or disappears after stratification for Y402H, but this SNP has no effect on the PLEKHA1, LOC387715, PRSS11/HTRA1, CCL28 or ADAM12. Similarly LOC3877156 SNP rs3750847 has no effect on association on chromosome 1 SNPs, although association with chromosome 10-associated SNPs disappears except for ADAM12. Thus, the ADAM12 association is not in LD with the previously established AMD locus on chromosome 10 (PLEKHA1, LOC387715, and PRSS11/HTRA1 genes). The ADAM12 signal appears to be coming from association with the over 84 group.

### INCORPORATION BY REFERENCE

The entire disclosure of each of the patent documents and scientific articles referred to herein is incorporated by reference for all purposes.

### Equivalents

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

TABLE 1
Risk-informative SNPs within or near C2, Factor B (BF), PLEKHA1, HTRA1, and PRELP

| | | | Allele Frequencies (percentages): | | | | | Allele Frequencies (percentages): | | | | | | |
|---|---|---|---|---|---|---|---|---|---|---|---|---|---|---|
| | | | Control Population | | | | | Disease Population | | | | | | Freq. Chi |
| | | | Homo- | | Het- | Al- | Al- | Homo- | | Het- | Al- | Al- | Genotype- | Square |
| | | Allele | zygotes | | ero- | lele 1 | lele 2 | zygotes | | ero- | lele 1 | lele 2 | Likelihood | (both col- |
| | | 1/ | Al- | Al- | zy- | Over- | Over- | Al- | Al- | zy- | Over- | Over- | Ratio (3 | lapsed - 2 |
| Gene | SNP | Allele 2 | lele 1 | lele 2 | gotes | all | all | lele 1 | lele 2 | gotes | all | all | categories) | categories) |
| C2 | rs1042663 | A/G | 1 | 82.1 | 16.9 | 9.5 | 90.5 | 0.4 | 87.9 | 11.7 | 6.2 | 93.8 | 6.54E−02 | 1.76E−02 |
| BF | rs4151670 | C/T | 92.9 | 0 | 7.1 | 96.5 | 3.5 | 96.6 | 0.2 | 3.2 | 98.2 | 1.8 | 2.84E−02 | 2.74E−02 |
| BF | rs4151650 | C/T | 98.6 | 0 | 1.4 | 99.3 | 0.7 | 99.8 | 0.2 | 0.0 | 99.8 | 0.2 | 1.11E−02 | 1.24E−01 |
| BF | rs4151671 | C/T | 90.2 | 0.7 | 9.2 | 94.7 | 5.3 | 94.8 | 0.2 | 5.0 | 97.3 | 2.7 | 4.16E−02 | 7.89E−03 |
| BF | rs4151672 | C/T | 90.2 | 0.7 | 9.1 | 94.8 | 5.2 | 94.9 | 0.2 | 5.0 | 97.3 | 2.7 | 4.22E−02 | 8.03E−03 |
| BF | rs550513 | A/G | 1 | 82.1 | 16.9 | 9.5 | 90.5 | 0.4 | 87.9 | 11.7 | 6.2 | 93.8 | 6.54E−02 | 1.76E−02 |
| PLEKHA1 | rs6585827 | A/G | 20.7 | 28.9 | 50.3 | 45.9 | 54.1 | 35.1 | 18.1 | 46.8 | 58.5 | 41.5 | 8.37E−06 | 1.25E−06 |
| PLEKHA1 | rs10887150 | A/C | 21.4 | 29 | 49.7 | 46.2 | 53.8 | 35.4 | 18.0 | 46.5 | 58.7 | 41.3 | 1.15E−05 | 1.45E−06 |
| PLEKHA1 | rs2421018 | A/G | 37.8 | 15.9 | 46.3 | 61.0 | 39.0 | 47.9 | 10.3 | 41.8 | 68.8 | 31.2 | 7.39E−03 | 1.41E−03 |
| PLEKHA1 | rs10082476 | A/G | 56.4 | 5.7 | 37.8 | 75.3 | 24.7 | 66.5 | 4.0 | 29.5 | 81.3 | 18.7 | 1.65E−02 | 4.86E−03 |
| PLEKHA1 | rs10399971 | C/T | 2 | 74.4 | 23.5 | 13.8 | 86.2 | 1.0 | 82.2 | 16.8 | 9.4 | 90.6 | 2.88E−02 | 6.62E−03 |
| PLEKHA1 | rs17649042 | C/T | 74.6 | 2 | 23.4 | 86.3 | 13.7 | 82.1 | 1.0 | 16.9 | 90.6 | 9.4 | 3.50E−02 | 8.33E−03 |
| HTRA1 | rs4237540 | A/G | 28.7 | 22 | 49.3 | 53.4 | 46.6 | 37.2 | 15.0 | 47.7 | 61.1 | 38.9 | 1.06E−02 | 2.52E−03 |
| HTRA1 | rs2268345 | G/T | 60.3 | 4.3 | 35.4 | 78.0 | 22.0 | 67.6 | 2.5 | 29.9 | 82.5 | 17.5 | 8.94E−02 | 3.02E−02 |
| HTRA1 | rs878107 | C/T | 4.4 | 61.7 | 33.9 | 21.4 | 78.6 | 2.6 | 68.3 | 29.1 | 17.1 | 82.9 | 1.07E−01 | 3.64E−02 |
| PRELP | rs947367 | A/G | 27.7 | 33.8 | 38.5 | 47.0 | 53.0 | 22.4 | 26.8 | 50.8 | 47.8 | 52.2 | 3.35E−03 | 7.40E−01 |

TABLE 1A
Additional risk-informative SNPs within or near HTRA1 and LOC387715

| | | | Allele Frequencies (percentages): | | | | | Allele Frequencies (percentages): | | | | | | |
|---|---|---|---|---|---|---|---|---|---|---|---|---|---|---|
| | | | Control Population | | | | | Disease Population | | | | | | Freq. Chi |
| | | | | | | | Al- | | | | | Al- | Genotype- | Square |
| | | Allele | | | | | lele 2 | | | | | lele 2 | Likelihood | (both col- |
| | | 1/ | Homozygotes | | Hetero- | Allele 1 | Over- | Homozygotes | | Hetero- | Allele 1 | Over- | Ratio (3 | lapsed - 2 |
| Gene | SNP | Allele 2 | Allele 1 | Allele 2 | zygotes | Overall | all | Allele 1 | Allele 2 | zygotes | Overall | all | categories) | categories) |
| LOC387715 | rs3750847 | A/G | 3.4 | 63.5 | 33.1 | 19.9 | 80.1 | 20.4 | 36.2 | 43.4 | 42.1 | 57.9 | 2.17E−18 | 1.58E−19 |
| HTRA1 | rs2253755 | A/G | 51.4 | 8.1 | 40.5 | 71.6 | 28.4 | 35.0 | 20.0 | 45.0 | 57.5 | 42.5 | 1.62E−07 | 1.78E−08 |

TABLE 2A
Control population cases

| | | | | | Allele Frequencies: | | | Allele Frequencies (percentages): | | | | |
|---|---|---|---|---|---|---|---|---|---|---|---|---|
| | | Allele | Control | | Control Population | | | Control Population | | | | |
| | | 1/ | Undeter. | | Homozygotes | | Hetero- | Homozygotes | | Hetero- | Allele 1 | Allele 2 |
| Gene | SNP | Allele 2 | Freq. | Control N | Allele 1 | Allele 2 | zygotes | Allele 1 | Allele 2 | zygotes | Overall | Overall |
| C2 | rs1042663 | A/G | 0 | 296 | 3 | 243 | 50 | 1 | 82.1 | 16.9 | 9.5 | 90.5 |
| BF | rs4151670 | C/T | 0 | 296 | 275 | 0 | 21 | 92.9 | 0 | 7.1 | 96.5 | 3.5 |
| BF | rs4151650 | C/T | 6 | 290 | 286 | 0 | 4 | 98.6 | 0 | 1.4 | 99.3 | 0.7 |
| BF | rs4151671 | C/T | 1 | 295 | 266 | 2 | 27 | 90.2 | 0.7 | 9.2 | 94.7 | 5.3 |
| BF | rs4151672 | C/T | 0 | 296 | 267 | 2 | 27 | 90.2 | 0.7 | 9.1 | 94.8 | 5.2 |
| BF | rs550513 | A/G | 0 | 296 | 3 | 243 | 50 | 1 | 82.1 | 16.9 | 9.5 | 90.5 |
| PLEKHA1 | rs6585827 | A/G | 2 | 294 | 61 | 85 | 148 | 20.7 | 28.9 | 50.3 | 45.9 | 54.1 |
| PLEKHA1 | rs10887150 | A/C | 6 | 290 | 62 | 84 | 144 | 21.4 | 29 | 49.7 | 46.2 | 53.8 |
| PLEKHA1 | rs2421018 | A/G | 0 | 296 | 112 | 47 | 137 | 37.8 | 15.9 | 46.3 | 61.0 | 39.0 |
| PLEKHA1 | rs10082476 | A/G | 0 | 296 | 167 | 17 | 112 | 56.4 | 5.7 | 37.8 | 75.3 | 24.7 |
| PLEKHA1 | rs10399971 | C/T | 3 | 293 | 6 | 218 | 69 | 2 | 74.4 | 23.5 | 13.8 | 86.2 |
| PLEKHA1 | rs17649042 | C/T | 1 | 295 | 220 | 6 | 69 | 74.6 | 2 | 23.4 | 86.3 | 13.7 |
| HTRA1 | rs4237540 | A/G | 0 | 296 | 85 | 65 | 146 | 28.7 | 22 | 49.3 | 53.4 | 46.6 |
| HTRA1 | rs2268345 | G/T | 19 | 277 | 167 | 12 | 98 | 60.3 | 4.3 | 35.4 | 78.0 | 22.0 |
| [symbol] | [symbol] | [symbol] | [symbol] | [symbol] | [symbol] | [symbol] | [symbol] | [symbol] | [symbol] | [symbol] | [symbol] | [symbol] |
| LOC387715 | rs3750847 | A/G | 0 | 296 | 10 | 188 | 98 | 3.4 | 63.5 | 33.1 | 19.9 | 80.1 |
| HTRA1 | rs2253755 | A/G | 0 | 296 | 152 | 24 | 120 | 51.4 | 8.1 | 40.5 | 71.6 | 28.4 |

[symbol]

indicates data missing or illegible when filed

TABLE 2B
Disease population cases

| | | | | | Allele Frequencies: | | | Allele Frequencies (percentages): | | | | |
|---|---|---|---|---|---|---|---|---|---|---|---|---|
| | | Allele | Disease | | Disease Population | | | Disease Population | | | | |
| | | 1/ | Undeter. | | Homozygotes | | Hetero- | Homozygotes | | Hetero- | Allele 1 | Allele 2 |
| Gene | SNP | Allele 2 | Freq. | Disease N | Allele 1 | Allele 2 | zygotes | Allele 1 | Allele 2 | zygotes | Overall | Overall |
| C2 | rs1042663 | A/G | 0 | 505 | 2 | 444 | 59 | 0.4 | 87.9 | 11.7 | 6.2 | 93.8 |
| BF | rs4151670 | C/T | 1 | 504 | 487 | 1 | 16 | 96.6 | 0.2 | 3.2 | 98.2 | 1.8 |
| BF | rs4151650 | C/T | 0 | 505 | 504 | 1 | 0 | 99.8 | 0.2 | 0.0 | 99.8 | 0.2 |
| BF | rs4151671 | C/T | 1 | 504 | 478 | 1 | 25 | 94.8 | 0.2 | 5.0 | 97.3 | 2.7 |
| BF | rs4151672 | C/T | 0 | 505 | 479 | 1 | 25 | 94.9 | 0.2 | 5.0 | 97.3 | 2.7 |
| BF | rs550513 | A/G | 0 | 505 | 2 | 444 | 59 | 0.4 | 87.9 | 11.7 | 6.2 | 93.8 |
| PLEKHA1 | rs6585827 | A/G | 3 | 502 | 176 | 91 | 235 | 35.1 | 18.1 | 46.8 | 58.5 | 41.5 |
| PLEKHA1 | rs10887150 | A/C | 0 | 505 | 179 | 91 | 235 | 35.4 | 18.0 | 46.5 | 58.7 | 41.3 |
| PLEKHA1 | rs2421018 | A/G | 0 | 505 | 242 | 52 | 211 | 47.9 | 10.3 | 41.8 | 68.8 | 31.2 |
| PLEKHA1 | rs10082476 | A/G | 3 | 502 | 334 | 20 | 148 | 66.5 | 4.0 | 29.5 | 81.3 | 18.7 |
| PLEKHA1 | rs10399971 | C/T | 0 | 505 | 5 | 415 | 85 | 1.0 | 82.2 | 16.8 | 9.4 | 90.6 |
| PLEKHA1 | rs17649042 | C/T | 2 | 503 | 413 | 5 | 85 | 82.1 | 1.0 | 16.9 | 90.6 | 9.4 |
| HTRA1 | rs4237540 | A/G | 0 | 505 | 188 | 76 | 241 | 37.2 | 15.0 | 47.7 | 61.1 | 38.9 |
| HTRA1 | rs2268345 | G/T | 27 | 478 | 323 | 12 | 143 | 67.6 | 2.5 | 29.9 | 82.5 | 17.5 |
| [symbol] | [symbol] | [symbol] | [symbol] | [symbol] | [symbol] | [symbol] | [symbol] | [symbol] | [symbol] | [symbol] | [symbol] | [symbol] |
| LOC387715 | rs3750847 | A/G | 0 | 505 | 103 | 183 | 219 | 20.4 | 36.2 | 43.4 | 42.1 | 57.9 |
| HTRA1 | rs2253755 | A/G | 5 | 500 | 175 | 100 | 225 | 35.0 | 20.0 | 45.0 | 57.5 | 42.5 |

[symbol]

indicates data missing or illegible when filed

TABLE 2C
Differences in genotype frequencies between cases and controls

| | | | Difference in | Difference in | Difference in | Difference in |
|---|---|---|---|---|---|---|
| | | | Percentage | Percentage Allele | Percentage | Percentage |
| | | Allele 1/ | Allele Frequency | Frequency | Allele Frequency | Allele Frequency |
| Gene | SNP | Allele 2 | (Allele 1) | (Hetero-Both) | (Allele 2) | (Undetermined) |
| C2 | rs1042663 | A/G | 0.6 | 5.2 | 5.8 | 0.0 |
| BF | rs4151670 | C/T | 3.7 | 3.9 | 0.2 | 0.2 |
| BF | rs4151650 | C/T | 1.2 | 1.4 | 0.2 | 2.0 |
| BF | rs4151671 | C/T | 4.6 | 4.2 | 0.5 | 0.1 |
| BF | rs4151672 | C/T | 4.7 | 4.1 | 0.5 | 0.0 |
| BF | rs550513 | A/G | 0.6 | 5.2 | 5.8 | 0.0 |
| PLEKHA1 | rs6585827 | A/G | 14.4 | 3.5 | 10.8 | 0.1 |
| PLEKHA1 | rs10887150 | A/C | 14.0 | 3.2 | 11.0 | 2.0 |
| PLEKHA1 | rs2421018 | A/G | 10.1 | 4.5 | 5.6 | 0.0 |
| PLEKHA1 | rs10082476 | A/G | 10.1 | 8.3 | 1.7 | 0.6 |
| PLEKHA1 | rs10399971 | C/T | 1.0 | 6.7 | 7.8 | 1.0 |
| PLEKHA1 | rs17649042 | C/T | 7.5 | 6.5 | 1.0 | 0.1 |
| PRSS11 | rs4237540 | A/G | 8.5 | 1.6 | 7.0 | 0.0 |
| PRSS11 | rs2268345 | G/T | 7.3 | 5.5 | 1.8 | 1.1 |
| [symbol] | [symbol] | [symbol] | [symbol] | [symbol] | [symbol] | [symbol] |
| LOC387715 | rs3750847 | A/G | 17.0 | 10.3 | 27.3 | 0.0 |
| PRSS11 | rs2253755 | A/G | 16.4 | 4.5 | 11.9 | 1.0 |

[symbol]

indicates data missing or illegible when filed

TABLE 3
Risk-informative SNPs in the RCA locus

| | | | | | | | | | | | | | | Fre- |
|---|---|---|---|---|---|---|---|---|---|---|---|---|---|---|
| | | | | | | | | | | | | | | quencies |
| | | | | | | | | | | | | | | Chi |
| | | Al- | Allele Frequencies (percentages): | | | | | Allele Frequencies (percentages): | | | | | Genotype- | Square |
| | | lele | Control Population | | | | | Disease Population | | | | | Likelihood | (both col- |
| | | 1/Al- | Homozygotes | | Hetero- | Allele 1 | Allele 2 | Homozygotes | | Hetero- | Allele 1 | Allele 2 | Ratio (3 | lapsed - 2 |
| Gene | SNP | lele 2 | Allele 1 | Allele 2 | zygotes | Overall | Overall | Allele 1 | Allele 2 | zygotes | Overall | Overall | categories) | categories) |
| F13B | rs5997 | A/G | 1 | 77.9 | 21 | 11.6 | 88.4 | 0.4 | 90.1 | 9.5 | 5.2 | 94.8 | 2.48E−05 | 3.37E−06 |
| F13B | rs6428380 | A/G | 1 | 78.4 | 20.6 | 11.3 | 88.7 | 0.4 | 90.1 | 9.5 | 5.2 | 94.8 | 4.11E−05 | 5.81E−06 |
| F13B | rs1412631 | C/T | 78.4 | 1 | 20.6 | 88.7 | 11.3 | 90.1 | 0.4 | 9.5 | 94.8 | 5.2 | 4.11E−05 | 5.81E−06 |
| F13B | rs1794006 | C/T | 78.4 | 1 | 20.6 | 88.7 | 11.3 | 89.9 | 0.4 | 9.7 | 94.7 | 5.3 | 6.13E−05 | 8.87E−06 |
| F13B | rs10801586 | C/T | 69.6 | 2 | 28.4 | 83.8 | 16.2 | 82.2 | 1.4 | 16.4 | 90.4 | 9.6 | 2.43E−04 | 8.70E−05 |
| F13B | rs2990510 | G/T | 8.4 | 45.6 | 45.9 | 31.4 | 68.6 | 15.0 | 39.2 | 45.7 | 37.9 | 62.1 | 1.31E−02 | 8.67E−03 |
| FHR1 | rs12027476 | C/G | 0 | 63.6 | 36.4 | 18.2 | 81.8 | 0.0 | 78.2 | 21.8 | 10.9 | 89.1 | 1.24E−05 | 4.99E−05 |
| FHR1 | rs436719 | A/C | 46.6 | 0 | 53.4 | 73.3 | 26.7 | 58.8 | 0.0 | 41.2 | 79.4 | 20.6 | 8.32E−04 | 5.04E−03 |
| FHR2 | rs12066959 | A/G | 5.5 | 58.7 | 35.8 | 23.4 | 76.6 | 2.0 | 75.0 | 23.0 | 13.5 | 86.5 | 4.83E−06 | 4.38E−07 |
| FHR2 | rs3828032 | A/G | 8.2 | 46.3 | 45.6 | 31.0 | 69.0 | 5.0 | 62.7 | 32.3 | 21.1 | 78.9 | 3.29E−05 | 1.16E−05 |
| FHR2 | rs6674522 | C/G | 1.4 | 76.7 | 22 | 12.3 | 87.7 | 0.4 | 87.9 | 11.7 | 6.2 | 93.8 | 1.79E−04 | 2.40E−05 |
| FHR2 | rs432366 | C/G | 0 | 47 | 53 | 26.5 | 73.5 | 0.0 | 58.8 | 41.2 | 20.6 | 79.4 | 1.15E−03 | 6.34E−03 |
| FHR4 | rs1409153 | A/G | 36.1 | 14.9 | 49 | 60.6 | 39.4 | 17.0 | 36.8 | 46.1 | 40.1 | 59.9 | 3.25E−14 | 1.93E−15 |
| FHR5 | rs10922153 | G/T | 23.6 | 25.7 | 50.7 | 49.0 | 51.0 | 44.6 | 9.5 | 45.9 | 67.5 | 32.5 | 1.38E−12 | 2.27E−13 |
| FHR5 | MRD_3905 | A/G | 3 | 57.8 | 39.2 | 22.6 | 77.4 | 3.4 | 68.9 | 27.7 | 17.2 | 82.8 | 3.74E−03 | 8.03E−03 |
| FHR5 | MRD_3906 | C/T | 57.8 | 3.7 | 38.5 | 77.0 | 23.0 | 68.5 | 3.4 | 28.1 | 82.6 | 17.4 | 8.16E−03 | 6.81E−03 |

TABLE 4
Risk-informative SNP in or near other genes

| | | | | | | | | | | | | | | Fre- |
|---|---|---|---|---|---|---|---|---|---|---|---|---|---|---|
| | | | | | | | | | | | | | | quencies |
| | | | Allele Frequencies (percentages): | | | | | Allele Frequencies (percentages): | | | | | | Chi |
| | | | Control Population | | | | | Disease Population | | | | | Genotype- | Square |
| | | Allele | Homozygotes | | | | | Homozygotes | | | | | Likelihood | (both col- |
| | | 1/ | Al- | Al- | Hetero- | Allele 1 | Allele 2 | Al- | Al- | Hetero- | Allele 1 | Allele 2 | Ratio (3 | lapsed-2 |
| Gene | SNP | Allele 2 | lele 1 | lele 2 | zygotes | Overall | Overall | lele 1 | lele 2 | zygotes | Overall | Overall | categories) | categories) |
| ADAM12 | rs1676717 | A/G | 17.6 | 29 | 53.4 | 44.3 | 55.7 | 13.5 | 41.2 | 45.3 | 36.1 | 63.9 | 2.16E−03 | 1.31E−03 |
| ADAM12 | rs1621212 | C/T | 29.7 | 17.2 | 53 | 56.3 | 43.8 | 40.8 | 13.5 | 45.7 | 63.7 | 36.3 | 6.13E−03 | 3.33E−03 |
| ADAM12 | rs12779767 | C/T | 41.9 | 10.8 | 47.3 | 65.5 | 34.5 | 34.7 | 15.4 | 49.9 | 59.6 | 40.4 | 5.33E−02 | 1.83E−02 |
| ADAM12 | rs11244834 | C/T | 10.8 | 41.4 | 47.8 | 34.7 | 65.3 | 15.4 | 34.7 | 49.9 | 40.4 | 59.6 | 6.87E−02 | 2.49E−02 |
| ADAM19 | rs12189024 | A/G | 6.4 | 59.1 | 34.5 | 23.6 | 76.4 | 10.1 | 48.3 | 41.6 | 30.9 | 69.1 | 8.23E−03 | 1.88E−03 |
| ADAM19 | rs7725839 | A/C | 2 | 75.3 | 22.6 | 13.3 | 86.7 | 4.4 | 66.9 | 28.8 | 18.8 | 81.3 | 2.06E−02 | 5.18E−03 |
| ADAM19 | rs11740315 | A/G | 8.1 | 58.1 | 33.7 | 25.0 | 75.0 | 10.5 | 47.5 | 42.0 | 31.5 | 68.5 | 2.61E−02 | 1.05E−02 |
| ADAM19 | rs7719224 | C/T | 74.9 | 2 | 23.1 | 86.4 | 13.6 | 67.1 | 4.4 | 28.5 | 81.4 | 18.6 | 3.24E−02 | 9.00E−03 |
| ADAM19 | rs6878446 | A/G | 9.5 | 54.1 | 36.5 | 27.7 | 72.3 | 11.5 | 45.3 | 43.2 | 33.1 | 66.9 | 5.85E−02 | 2.51E−02 |
| APBA2 | rs3829467 | C/T | 0.3 | 84.9 | 14.7 | 7.7 | 92.3 | 1.8 | 78.9 | 19.3 | 11.5 | 88.5 | 3.25E−02 | 1.67E−02 |
| APOB | rs12714097 | C/T | 98.6 | 0 | 1.4 | 99.3 | 0.7 | 100.0 | 0.0 | 0.0 | 100.0 | 0.0 | 4.68E−03 | 8.91E−03 |
| BMP7 | rs6014959 | A/G | 83.4 | 1.4 | 15.3 | 91.0 | 9.0 | 75.8 | 1.6 | 22.6 | 87.1 | 12.9 | 3.51E−02 | 1.77E−02 |
| BMP7 | rs6064517 | C/T | 83.8 | 1 | 15.2 | 91.4 | 8.6 | 76.4 | 1.6 | 22.0 | 87.4 | 12.6 | 4.31E−02 | 1.49E−02 |
| BMP7 | rs162315 | A/G | 5.1 | 64.5 | 30.4 | 20.3 | 79.7 | 6.9 | 56.0 | 37.0 | 25.4 | 74.6 | 5.71E−02 | 1.84E−02 |
| BMP7 | rs162316 | A/G | 5.1 | 64.5 | 30.4 | 20.3 | 79.7 | 6.7 | 56.0 | 37.2 | 25.3 | 74.7 | 5.89E−02 | 2.07E−02 |
| C1NH | rs4926 | A/G | 4.7 | 56.8 | 38.5 | 24.0 | 76.0 | 8.1 | 45.9 | 45.9 | 31.1 | 68.9 | 6.66E−03 | 2.36E−03 |
| C1 [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| C1 [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| C1Qa | rs172376 | A/G | 34.9 | 18.6 | 46.4 | 58.1 | 41.9 | 42.1 | 13.3 | 44.5 | 64.4 | 35.6 | 4.93E−02 | 1.26E−02 |
| C1RL | rs61917913 | A/G | 0 | 94.9 | 5.1 | 2.5 | 97.5 | 0.0 | 91.1 | 8.9 | 4.5 | 95.5 | 3.97E−02 | 4.97E−02 |
| C4BPA | rs2842706 | A/G | 98.9 | 0 | 1.1 | 99.4 | 0.6 | 100.0 | 0.0 | 0.0 | 100.0 | 0.0 | 1.37E−02 | 2.20E−02 |
| C4BPA | rs1126618 | C/T | 63.5 | 2.4 | 34.1 | 80.6 | 19.4 | 71.4 | 2.2 | 26.4 | 84.6 | 15.4 | 6.43E−02 | 3.68E−02 |
| C5 | rs7033790 | C/T | 68.6 | 3 | 28.4 | 82.8 | 17.2 | 62.2 | 7.3 | 30.5 | 77.4 | 22.6 | 1.80E−02 | 1.07E−02 |
| C5 | rs10739585 | C/G | 68.6 | 3 | 28.4 | 82.8 | 17.2 | 62.2 | 7.3 | 30.5 | 77.4 | 22.6 | 1.80E−02 | 1.07E−02 |
| C5 | rs2230214 | A/G | 2 | 75.3 | 22.6 | 13.3 | 86.7 | 1.4 | 82.8 | 15.8 | 9.3 | 90.7 | 4.22E−02 | 1.20E−02 |
| C5 | rs10985127 | A/G | 61.3 | 4.8 | 33.9 | 78.3 | 21.7 | 69.9 | 3.2 | 26.9 | 83.3 | 16.7 | 4.42E−02 | 1.20E−02 |
| C5 | rs2300932 | A/C | 12.5 | 43.2 | 44.3 | 34.6 | 65.4 | 17.2 | 35.8 | 46.9 | 40.7 | 59.3 | 5.84E−02 | 1.60E−02 |
| C5 | rs10985126 | C/T | 4.7 | 61.8 | 33.4 | 21.5 | 78.5 | 3.2 | 69.9 | 26.9 | 16.6 | 83.4 | 5.86E−02 | 1.63E−02 |
| C5 | rs12683026 | A/G | 78.4 | 1.7 | 19.9 | 88.3 | 11.7 | 84.6 | 0.8 | 14.7 | 91.9 | 8.1 | 7.39E−02 | 1.94E−02 |
| C5 | rs3815467 | A/G | 4.7 | 62.5 | 32.8 | 21.1 | 78.9 | 3.2 | 70.1 | 26.7 | 16.5 | 83.5 | 7.77E−02 | 2.19E−02 |
| C5 | rs4837805 | A/G | 43.2 | 11.5 | 45.3 | 65.9 | 34.1 | 37.2 | 15.8 | 46.9 | 60.7 | 39.3 | 1.13E−01 | 3.84E−02 |
| C8A | MRD_4048 | C/G | 99.7 | 0 | 0.3 | 99.8 | 0.2 | 97.4 | 0.0 | 2.6 | 98.7 | 1.3 | 8.80E−03 | 2.04E−02 |
| C8A | MRD_4044 | A/C | 0 | 99.7 | 0.3 | 0.2 | 99.8 | 0.0 | 97.4 | 2.6 | 1.3 | 98.7 | 9.03E−03 | 2.08E−02 |
| C9 | rs476569 | C/T | 23.6 | 25 | 51.4 | 49.3 | 50.7 | 31.9 | 19.2 | 48.9 | 56.3 | 43.7 | 2.23E−02 | 6.59E−03 |
| CCL28 | rs7380703 | G/T | 4.1 | 62.8 | 33.1 | 20.6 | 79.4 | 10.1 | 50.2 | 39.7 | 30.0 | 70.0 | 1.87E−04 | 4.27E−05 |
| CCL28 | rs11741246 | A/G | 27 | 23.6 | 49.3 | 51.7 | 48.3 | 22.4 | 31.5 | 46.0 | 45.4 | 54.6 | 4.46E−02 | 1.56E−02 |
| CCL28 | rs4443426 | C/T | 24.3 | 27 | 48.6 | 48.6 | 51.4 | 31.5 | 22.0 | 46.5 | 54.8 | 45.2 | 6.27E−02 | 1.82E−02 |
| CLU | MRD_4452 | A/G | 0 | 98 | 2 | 1.0 | 99.0 | 0.0 | 94.7 | 5.3 | 2.7 | 97.3 | 1.62E−02 | 2.40E−02 |
| COL9A1 | rs1135056 | A/G | 28.4 | 17.6 | 54.1 | 55.4 | 44.6 | 38.3 | 16.9 | 44.8 | 60.7 | 39.3 | 1.27E−02 | 3.73E−02 |
| FGFR2 | rs2981582 | C/T | 31.8 | 19.6 | 48.6 | 56.1 | 43.9 | 41.6 | 13.7 | 44.8 | 64.0 | 36.0 | 8.59E−03 | 1.80E−03 |
| FGFR2 | rs2912774 | A/C | 20.6 | 32.1 | 47.3 | 44.3 | 55.7 | 14.5 | 40.6 | 45.0 | 36.9 | 63.1 | 1.82E−02 | 3.81E−03 |
| FGFR2 | rs1319093 | A/T | 2.7 | 66.7 | 30.6 | 18.0 | 82.0 | 2.4 | 74.3 | 23.4 | 14.1 | 85.9 | 7.17E−02 | 3.46E−02 |
| FGFR2 | rs10510088 | A/G | 59.1 | 4.4 | 36.5 | 77.4 | 22.6 | 67.1 | 3.8 | 29.1 | 81.7 | 18.3 | 7.41E−02 | 3.67E−02 |
| FG [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| HA [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| HABP2 | rs7080536 | A/G | 0 | 95.2 | 4.8 | 2.4 | 97.6 | 0.2 | 90.9 | 8.9 | 4.7 | 95.3 | 4.99E−02 | 2.14E−02 |
| EMID2 | rs17135580 | C/T | 0.7 | 79 | 20.3 | 10.8 | 89.2 | 2.4 | 70.9 | 26.7 | 15.7 | 84.3 | 1.51E−02 | 6.38E−03 |
| EMID2 | rs12536189 | C/T | 0.7 | 79.1 | 20.3 | 10.8 | 89.2 | 2.4 | 71.0 | 26.6 | 15.7 | 84.3 | 1.55E−02 | 6.58E−03 |
| EMID2 | rs7778986 | A/G | 1.4 | 75.6 | 23 | 12.9 | 87.1 | 2.7 | 68.2 | 29.2 | 17.2 | 82.8 | 6.35E−02 | 2.18E−02 |
| EMID2 | rs11766744 | A/G | 1.7 | 78.6 | 19.7 | 11.5 | 88.5 | 2.2 | 71.8 | 26.0 | 15.2 | 84.8 | 9.59E−02 | 3.97E−02 |
| COL6A3 | rs4663722 | C/G | 81.4 | 2 | 16.6 | 89.7 | 10.3 | 86.5 | 0.6 | 12.9 | 92.9 | 7.1 | 6.42E−02 | 2.28E−02 |
| COL6A3 | rs1874573 | A/G | 48 | 9.8 | 42.2 | 69.1 | 30.9 | 36.4 | 12.5 | 51.1 | 62.0 | 38.0 | 5.84E−03 | 4.09E−03 |
| COL6A3 | rs12992087 | C/T | 68.9 | 0.3 | 30.7 | 84.3 | 15.7 | 65.9 | 3.4 | 30.7 | 81.3 | 18.7 | 6.10E−03 | 1.28E−01 |
| CH21 | rs2826552 | A/T | 11.1 | 46.7 | 42.1 | 32.2 | 67.8 | 12.4 | 35.4 | 52.2 | 38.5 | 61.5 | 9.90E−03 | 1.55E−02 |
| COL4A1 | rs7338606 | C/T | 56.8 | 5.7 | 37.5 | 75.5 | 24.5 | 68.1 | 3.6 | 28.3 | 82.3 | 17.7 | 4.86E−03 | 1.13E−03 |
| COL4A1 | rs11842143 | C/G | 9.5 | 52 | 38.5 | 28.7 | 71.3 | 13.9 | 41.0 | 45.1 | 36.4 | 63.6 | 6.83E−03 | 1.59E−03 |
| COL4A1 | rs595325 | G/T | 4.4 | 72.3 | 23.3 | 16.0 | 84.0 | 5.6 | 63.3 | 31.2 | 21.1 | 78.9 | 3.14E−02 | 1.28E−02 |
| COL4A1 | rs9301441 | C/T | 16.2 | 40.5 | 43.2 | 37.8 | 62.2 | 20.6 | 31.7 | 47.7 | 44.5 | 55.5 | 3.24E−02 | 9.59E−03 |
| COL4A1 | rs754880 | A/G | 14.9 | 34.5 | 50.7 | 40.2 | 59.8 | 21.6 | 29.5 | 48.9 | 46.0 | 54.0 | 4.65E−02 | 2.31E−02 |
| COL4A1 | rs7139492 | C/T | 50.3 | 8.6 | 41.1 | 70.9 | 29.1 | 58.8 | 5.9 | 35.2 | 76.4 | 23.6 | 5.29E−02 | 1.45E−02 |
| COL4A1 | rs72509 | G/T | 3.4 | 67.9 | 28.7 | 17.7 | 82.3 | 2.2 | 74.5 | 23.4 | 13.9 | 86.1 | 1.23E−01 | 3.75E−02 |
| FBLN2 | rs9843344 | A/G | 13.9 | 37.5 | 48.6 | 38.2 | 61.8 | 10.1 | 46.5 | 43.4 | 31.8 | 68.2 | 3.06E−02 | 9.19E−03 |
| FBLN2 | rs1562808 | C/T | 41.8 | 10.2 | 48 | 65.8 | 34.2 | 50.0 | 7.1 | 42.9 | 71.4 | 28.6 | 5.51E−02 | 1.90E−02 |
| FBN2 | rs10057855 | A/G | 1.7 | 85.8 | 12.5 | 7.9 | 92.1 | 1.4 | 76.0 | 22.6 | 12.7 | 87.3 | 1.49E−03 | 3.37E−03 |
| FBN2 | rs10057405 | A/C | 82.4 | 1.7 | 15.9 | 90.4 | 9.6 | 72.5 | 1.8 | 25.7 | 85.3 | 14.7 | 4.00E−03 | 3.66E−03 |
| FBN2 | rs331075 | A/G | 36.5 | 13.2 | 50.3 | 61.7 | 38.3 | 27.7 | 20.8 | 51.5 | 53.5 | 46.5 | 4.32E−03 | 1.42E−03 |
| FBN2 | rs17676236 | C/G | 2 | 81.4 | 16.6 | 10.3 | 89.7 | 1.6 | 72.7 | 25.7 | 14.5 | 85.5 | 8.92E−03 | 1.68E−02 |
| FBN2 | rs6891153 | C/T | 1.4 | 87.8 | 10.8 | 6.8 | 93.2 | 0.8 | 80.6 | 18.7 | 10.1 | 89.9 | 8.93E−03 | 2.24E−02 |
| FBN2 | rs17676260 | C/T | 2 | 81.1 | 16.9 | 10.5 | 89.5 | 1.6 | 72.5 | 25.9 | 14.6 | 85.4 | 1.07E−02 | 1.92E−02 |
| FBN2 | rs154001 | C/T | 10.8 | 51.4 | 37.8 | 29.7 | 70.3 | 13.7 | 40.6 | 45.7 | 36.5 | 63.5 | 1.25E−02 | 5.52E−03 |
| FB [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| FB [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| FBN2 | rs3828661 | A/C | 63.1 | 3.1 | 33.8 | 80.0 | 20.0 | 54.9 | 4.8 | 40.4 | 75.0 | 25.0 | 5.88E−02 | 2.28E−02 |
| FBN2 | rs3828661 | A/C | 63.1 | 3.1 | 33.8 | 80.0 | 20.0 | 54.9 | 4.8 | 40.4 | 75.0 | 25.0 | 5.88E−02 | 2.28E−02 |
| FBN2 | rs11241955 | A/G | 10.8 | 42.6 | 46.6 | 34.1 | 65.9 | 7.7 | 49.9 | 42.4 | 28.9 | 71.1 | 8.74E−02 | 2.93E−02 |
| FBN2 | rs6882394 | C/T | 6.6 | 50.3 | 43.1 | 28.1 | 71.9 | 9.9 | 44.1 | 46.1 | 32.9 | 67.1 | 1.20E−01 | 4.91E−02 |
| FBN2 | rs432792 | C/T | 1.7 | 69.6 | 28.7 | 16.0 | 84.0 | 1.2 | 76.2 | 22.6 | 12.5 | 87.5 | 1.20E−01 | 4.54E−02 |
| FBN2 | rs13181926 | C/T | 62.5 | 3.4 | 34.1 | 79.6 | 20.4 | 56.4 | 5.7 | 37.8 | 75.3 | 24.7 | 1.27E−01 | 5.34E−02 |
| FCN1 | rs10117466 | G/T | 50.2 | 8.8 | 41 | 70.7 | 29.3 | 39.2 | 12.2 | 48.6 | 63.5 | 36.5 | 9.29E−03 | 3.66E−03 |
| FCN1 | rs10120023 | C/T | 46.6 | 9.1 | 44.3 | 68.8 | 31.3 | 36.8 | 13.3 | 49.9 | 61.8 | 38.2 | 1.47E−02 | 4.95E−03 |
| FCN1 | rs7857015 | A/G | 46.3 | 9.1 | 44.6 | 68.6 | 31.4 | 36.8 | 13.3 | 49.9 | 61.8 | 38.2 | 1.83E−02 | 6.12E−03 |
| FCN1 | rs2989727 | C/T | 17.9 | 35.8 | 46.3 | 41.0 | 59.0 | 12.9 | 43.0 | 44.2 | 35.0 | 65.0 | 5.69E−02 | 1.48E−02 |
| FCN1 | rs1071583 | C/T | 37.8 | 17.2 | 44.9 | 60.3 | 39.7 | 43.1 | 11.7 | 45.2 | 65.7 | 34.3 | 7.15E−02 | 3.10E−02 |
| FCN1 | rs3012788 | C/T | 68.9 | 0.7 | 30.3 | 84.1 | 15.9 | 60.7 | 1.3 | 38.0 | 79.7 | 20.3 | 7.65E−02 | 3.91E−02 |
| HS3ST4 | rs4441276 | A/G | 43.2 | 7.1 | 49.7 | 68.1 | 31.9 | 49.2 | 12.1 | 38.7 | 68.6 | 31.4 | 3.35E−03 | 8.43E−01 |
| HS3ST4 | rs12921387 | C/T | 6.8 | 51.2 | 42 | 27.8 | 72.2 | 11.5 | 45.7 | 42.7 | 32.9 | 67.1 | 5.76E−02 | 3.33E−02 |
| IGLC1 | rs1065464 | C/G | 1.4 | 77.7 | 20.9 | 11.8 | 88.2 | 0.0 | 72.9 | 27.1 | 13.5 | 86.5 | 3.33E−03 | 3.22E−01 |
| IGLC1 | rs4820495 | C/T | 50.7 | 9.8 | 39.5 | 70.4 | 29.6 | 42.4 | 11.3 | 46.3 | 65.5 | 34.5 | 7.49E−02 | 4.37E−02 |
| IL12RB1 | rs273493 | C/T | 92.6 | 0 | 7.4 | 96.3 | 3.7 | 86.8 | 0.0 | 13.2 | 93.4 | 6.6 | 1.14E−02 | 1.69E−02 |
| ITGA4 | rs3770115 | C/T | 40.2 | 12.5 | 47.3 | 63.9 | 36.1 | 51.9 | 9.7 | 38.4 | 71.1 | 28.9 | 5.83E−03 | 2.63E−03 |
| ITGA4 | rs4667319 | A/G | 38.9 | 14.2 | 47 | 62.3 | 37.7 | 48.1 | 11.7 | 40.2 | 68.2 | 31.8 | 3.79E−02 | 1.63E−02 |
| ITGAX | rs2230429 | C/G | 47.8 | 8.1 | 44.1 | 69.8 | 30.2 | 42.9 | 14.9 | 42.3 | 64.0 | 36.0 | 1.48E−02 | 1.72E−02 |
| ITGAX | rs11574630 | C/T | 49 | 7.8 | 43.2 | 70.6 | 29.4 | 42.8 | 13.9 | 43.4 | 64.5 | 35.5 | 1.91E−02 | 1.16E−02 |
| MASP1 | rs12638131 | G/T | 49.3 | 8.4 | 42.2 | 70.4 | 29.6 | 57.9 | 7.1 | 34.9 | 75.4 | 24.6 | 6.14E−02 | 2.99E−02 |
| MASP2 | rs12142107 | C/T | 94.9 | 0 | 5.1 | 97.4 | 2.6 | 97.8 | 0.0 | 2.2 | 98.9 | 1.1 | 2.81E−02 | 2.60E−02 |
| MYOC | rs2236875 | G/T | 79.7 | 2 | 18.2 | 88.9 | 11.1 | 85.9 | 0.2 | 13.9 | 92.9 | 7.1 | 5.92E−03 | 5.64E−03 |
| MYOC | rs12035960 | C/T | 80.1 | 2 | 17.9 | 89.0 | 11.0 | 85.9 | 0.2 | 13.9 | 92.9 | 7.1 | 7.27E−03 | 7.80E−03 |
| M [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| PP [symbol] | [symbol] | [symbol] | [symbol] | [symbol] | [symbol] | [symbol] | [symbol] | [symbol] | [symbol] | [symbol] | [symbol] | [symbol] | [symbol] | [symbol] |
| PPID | rs7689418 | G/T | 58.1 | 6.4 | 35.5 | 75.8 | 24.2 | 46.6 | 9.1 | 44.2 | 68.8 | 31.3 | 6.52E−03 | 2.44E−03 |
| PTPRC | rs1932433 | C/T | 35.4 | 17.7 | 46.9 | 58.8 | 41.2 | 42.0 | 9.6 | 48.4 | 66.2 | 33.8 | 3.08E−03 | 3.11E−03 |
| PTPRC | rs17670373 | A/G | 48.6 | 12.2 | 39.2 | 68.2 | 31.8 | 37.2 | 12.3 | 50.5 | 62.5 | 37.5 | 4.02E−03 | 1.98E−02 |
| PTPRC | rs10919560 | A/G | 29.2 | 20.3 | 50.5 | 54.4 | 45.6 | 22.4 | 24.6 | 53.0 | 48.9 | 51.1 | 8.08E−02 | 3.39E−02 |
| SLC2A2 | rs7646014 | C/G | 2 | 74 | 24 | 14.0 | 86.0 | 0.4 | 82.4 | 17.2 | 9.0 | 91.0 | 4.79E−03 | 1.87E−03 |
| SLC2A2 | rs1604038 | C/T | 46.6 | 8.8 | 44.6 | 68.9 | 31.1 | 56.7 | 6.2 | 37.1 | 75.3 | 24.7 | 1.81E−02 | 5.56E−03 |
| SLC2A2 | rs5400 | C/T | 74.3 | 2 | 23.6 | 86.1 | 13.9 | 81.8 | 0.6 | 17.6 | 90.6 | 9.4 | 1.91E−02 | 6.15E−03 |
| SLC2A2 | rs11721319 | A/G | 2 | 74.7 | 23.3 | 13.7 | 86.3 | 0.6 | 81.7 | 17.7 | 9.4 | 90.6 | 2.48E−02 | 8.59E−03 |
| SPOCK | rs1229729 | A/G | 31.4 | 24.3 | 44.3 | 53.5 | 46.5 | 33.5 | 14.9 | 51.7 | 59.3 | 40.7 | 3.70E−03 | 2.45E−02 |
| SPOCK | rs1229731 | A/G | 24.3 | 31.1 | 44.6 | 46.6 | 53.4 | 14.9 | 33.5 | 51.7 | 40.7 | 59.3 | 3.91E−03 | 2.07E−02 |
| SPOCK | rs2961633 | A/G | 19.7 | 32.9 | 47.5 | 43.4 | 56.6 | 11.6 | 37.4 | 51.0 | 37.1 | 62.9 | 8.54E−03 | 1.32E−02 |
| SPOCK | rs2961632 | C/T | 33.7 | 18.7 | 47.6 | 57.5 | 42.5 | 37.8 | 11.5 | 50.7 | 63.2 | 36.8 | 1.95E−02 | 2.46E−02 |
| SPOCK | rs12656717 | A/G | 18.9 | 29.4 | 51.7 | 44.8 | 55.2 | 25.0 | 22.0 | 53.1 | 51.5 | 48.5 | 2.74E−02 | 9.39E−03 |
| TGFBR2 | rs4955212 | C/T | 52 | 9.8 | 38.2 | 71.1 | 28.9 | 60.4 | 5.7 | 33.9 | 77.3 | 22.7 | 2.51E−02 | 5.56E−03 |
| TGFBR2 | rs1019855 | C/T | 0.3 | 80.7 | 19 | 9.8 | 90.2 | 1.8 | 74.7 | 23.6 | 13.6 | 86.4 | 3.93E−02 | 2.76E−02 |
| TGFBR2 | rs2082225 | A/G | 80.3 | 0.3 | 19.3 | 90.0 | 10.0 | 74.7 | 1.8 | 23.6 | 86.4 | 13.6 | 4.72E−02 | 3.59E−02 |
| TGFBR2 | rs9823731 | A/G | 16.9 | 35.8 | 47.3 | 40.5 | 59.5 | 13.1 | 42.2 | 44.8 | 35.4 | 64.6 | 1.33E−01 | 4.18E−02 |

[symbol]

indicates data missing or illegible when filed

TABLE 5

| GENE NAME | GENE ID |
|---|---|
| C2 | ENSG00000166278 |
| FACTOR B | ENSG00000166285 |
| PLEKHA1 | ENSG00000107679 |
| HTRA1 | ENSG00000166033 |
| PRELP | ENSG00000188783 |

TABLE 6
Flanking Sequences for SNPs shown in Table 1

| Gene | SNP | SNP Flanking Sequence |
|---|---|---|
| C2 | rs1042663 | atgaaaatggaactgggactaacacctatgcNgccttaaacagtgtctatctcatgatgaaca |
| BF | rs4151670 | catttctgactctcccagactccttcatgtaNgacacccctcaagaggtggccgaagctttcc |
| BF | rs4151650 | ATGAGATCTCTTTCCACTGCTATGACGGTTANACTCTCCGGGGCTCTGCCAATCGCACCTGC |
| BF | rs4151671 | gagatgacagtggtgggagcagctgaagtgaNgcagtctattcgtccagaggaagagctgctc |
| BF | rs4151672 | tttctataaggggtttcctgctggacaggggNgtgggattgaattaaaacagctgcgacaaca |
| BF | rs550513 | AGAGGAAGGGGAAGAAACAGCTAGAGGCTTNAGAGAGAATGGTGAGGGCCAAAGCTACACC |
| PLEKHA1 | rs6585827 | GTGCTAACAACCAGTTCTGGTGAGGGGTATTCNATGAAATAAAATGTGTATGTGgttggtagg |
| PLEKHA1 | rs10887150 | GGAATGAAATATTTACATAGTTTCAAAGTANCTGTCTACTAAAATAGGTATTAAGTGTTGT |
| PLEKHA1 | rs2421018 | cagcctcttcaaatgagttgtaattttttgctNgtggagagttttaactcaatgttggtggct |
| PLEKHA1 | rs10082476 | TGTATGTGCACATGTGCTTTGCTTGATAAANGTACCTAGTCCCTAAAGGGGAATATAGAAA |
| PLEKHA1 | rs10399971 | GAGATTCTTGAAGACATATTTACATTTCTTNTCCTTCTTTAAAGTTAAAAACCAAAAACCC |
| PLEKHA1 | rs17649042 | ATGGTGGGGAACTTCCAAATGGAAATGTTNTGTTGACAGTAATCGAGGACTGGATGGAGCT |
| HTRA1 | rs4237540 | GCGGATAAGCTGCCGCTGACAGACCTGCCCNGTTTCTTAGCTCATCCCGGCCTCCATCCTG |
| HTRA1 | rs2268345 | GCGTTTGTTTACAGCTGTCTGGTGACATTCNCCAGGCTCTGTTTTCAGAAGGAACATTTCC |
| HTRA1 | rs878107 | TTGAAAGCAAAAATAATAATATGATACTGTNCTGAATTTGTTAAATTATTCTTCCAAGTAG |
| PRELP | rs947367 | TCCACCTTCTTCCCCAGGAGTCCTGAATCCNTGTGTTTCCAGGCCCTCAGAGCAGATGGCT |

TABLE 6A
Flanking Sequences for SNPs shown in Table 1A

| Gene | SNP | SNP Flanking Sequence |
|---|---|---|
| LOC387715 | rs3750847 | ACAATTCAAACAGAGCCCCAGGCAGCCACCNAAAGGTCTTGAATGACAGCTTGTCAATTTC |
| HTRA1 | rs2253755 | GGACTAATACAGTAGTGCAGTCATTTTTTCNTGGTCCCCAGTAAGGCCAAAAAATACCCAA |

1. A method of screening for susceptibility to complement dysregulation in an individual comprising screening for the presence or absence of a genetic profile characterized by polymorphisms in the genome of the individual associated with complement dysregulation, wherein the presence of a said genetic profile is indicative of the individual's risk of complement dysregulation, wherein the genetic profile comprises at least one polymorphism selected from Table 1 or Table 1A.

2. A method of determining an individual's risk of development or progression of age-related macular degeneration (AMD) comprising screening for the presence or absence of a genetic profile characterized by polymorphisms in the genome of the individual associated with risk for or protection against AMD, wherein the presence of a said genetic profile is indicative of the individual's relative risk of AMD, wherein the genetic profile comprises at least one polymorphism selected from Table 1 or Table 1A.

3. The method of claim 2, wherein the genetic profile comprises at least one polymorphism selected from Table 1.

4. A method of claim 2, comprising screening for at least two of said polymorphisms.

5. (canceled)

6. A method of claim 2, comprising screening for at least ten of said polymorphisms.

7. A method of claim 2, comprising screening for a combination of at least one predisposing polymorphism and at least one protective polymorphism.

8. A method of claim 2, comprising screening additionally for genomic deletions associated with AMD risk or AMD protection.

9. (canceled)

10. The method of claim 7, comprising screening for an additional polymorphism selected from the group consisting a polymorphism in exon 22 of CFH (R1210C), rs2511989, rs1061170, rs203674, rs1061147, rs2274700, rs12097550, rs203674, rs9427661, rs9427662, rs10490924, rs11200638, rs2230199, rs800292, rs3766404, rs529825, rs641153, rs4151667, rs547154, rs9332739, rs3753395, rs1410996, rs393955, rs403846, rs1329421, rs10801554, rs12144939, rs12124794, rs2284664, rs16840422, and rs6695321.

11. The method of claim 9, comprising screening for an additional polymorphism selected from Table 3.

12. The method of claim 9, comprising screening for an additional polymorphism selected from Table 4.

13. (canceled)

14. A method of claim 2, wherein the screening comprises analyzing a sample of said individual's DNA or RNA.

15. A method of claim 2, wherein the screening comprises analyzing a sample of said individual's proteome to detect an isoform encoded by an allelic variant in a protein thereof consequent of the presence of a said polymorphism in said individual's genome.

16. (canceled)

17. (canceled)

18. A method of claim 14, wherein said individual is determined to be at risk of developing AMD symptoms, comprising the additional step of prophylactically or therapeutically treating said individual to inhibit development thereof.

19. (canceled)

20. A method for treating or slowing the onset of AMD, the method comprising prophylactically or therapeutically treating an individual identified as having a genetic profile characterized by polymorphisms in the genome of the individual indicative of risk for developing AMD, wherein the presence of a said genetic profile is indicative of the individual's risk of developing AMD, wherein the genetic profile comprises at least one polymorphism selected from Table 1 or 1A.

21. The method of claim 20, wherein the genetic profile comprises at least one polymorphism selected from Table 1.

22. The method of claim 20, comprising administering a factor H polypeptide to the individual.

23. The method of claim 22 wherein the factor H polypeptide is encoded by a factor H protective haplotype.

24. A method according to claim 20, comprising inhibiting HTRA1 expression or activity in the individual.

25. The method of claim 24, comprising administering an antibody that binds HTRA1.

26. The method of claim 24, comprising administering a nucleic acid inhibiting HTRA1 expression or activity.

27.-29. (canceled)
